(19) United States
(12) Patent Application Publication
(10) Pub. No.: US 2016/0210410 A1
(43) Pub. Date: Jul. 21, 2016

(54) RADIOGRAPHIC IMAGING DEVICE, RADIOGRAPHIC IMAGING SYSTEM, IDENTIFICATION DATA APPLICATION METHOD, AND PROGRAM STORAGE MEDIUM

(71) Applicant: FUJIFILM CORPORATION, Tokyo (JP)

(72) Inventors: Yasufumi ODA, Kanagawa (JP); Ryo IMAMURA, Kanagawa (JP); Haruyasu NAKATSUGAWA, Kanagawa (JP)

(21) Appl. No.: 14/973,735

(22) Filed: Dec. 18, 2015

(30) Foreign Application Priority Data
Jan. 15, 2015 (JP) 2015-006123
Oct. 23, 2015 (JP) 2015-209216

(51) Int. Cl. G06F 19/00; G06F 17/30; A61B 6/00

(52) U.S. Cl. CPC G06F 19/321; A61B 6/463; G06F 19/322; G06F 17/30268; G06F 17/3028

(57) ABSTRACT

A radiographic imaging device includes an imaging unit that is configured to acquire a radiographic image in accordance with irradiated radiation; a storage unit that is configured to store sets of image data for plural radiographic images acquired by the imaging unit; and an identification data application unit that is configured to store identification data in the storage unit in a case in which a predetermined condition is detected, the identification data being used for partitioning, between before and after the detection of the predetermined condition, the sets of image data stored in the storage unit.

### CROSS-REFERENCE TO RELATED APPLICATION

This application claims priority under 35 USC 119 from Japanese Patent Applications No. 2015-006123 filed on Jan. 15, 2015, and No. 2015-209216 filed on Oct. 23, 2015, the disclosures of which are incorporated by reference herein.

### BACKGROUND

1. Field of the Invention

The present disclosure relates to a radiographic imaging device, a radiographic imaging system, an identification data application method, and a program storage medium.

2. Related Art

Conventionally, as a radiographic imaging device imaging a subject, for example, a radiographic imaging device that executes imaging process for the purpose of medical diagnosis is known. The radiographic imaging device detects radiation that has been emitted from a radiation irradiation device and transmitted through a subject by using a radiation detector, thereby imaging a radiographic image.

Technologies for storing image data of a radiographic image detected by a radiation detector inside a radiographic imaging device are known. For example, Japanese Patent Application Laid-Open (JP-A) No. 2010-190911 discloses a technology for storing various kinds of information such as information relating to radiographic imaging and subject information including name, sex and the like of a subject in a memory in association with image data of a radiation image.

The image data of a radiographic image stored in a radiographic imaging device needs to be associated with the imaged subject. However, in the technologies described above, there are cases in which it is difficult to associate the subject with the image data of the radiographic image. Particularly, it may be difficult to associate a subject and image data of a radiographic image with each other in a device other than a radiographic imaging device after the radiographic image has been acquired.

### SUMMARY

The present disclosure provides a radiographic imaging device, a radiographic imaging system, an identification data application method, and a program storage medium capable of associating a subject and image data of a corresponding radiographic image.

A first aspect of the present disclosure is a radiographic imaging device including: an imaging unit that is configured to acquire a radiographic image in accordance with irradiated radiation; a storage unit that is configured to store sets of image data for plural radiographic images acquired by the imaging unit; and an identification data application unit that is configured to store identification data in the storage unit in a case in which a predetermined condition is detected, the identification data being used for partitioning, between before and after the detection of the predetermined condition, the sets of image data stored in the storage unit.

In the first aspect, in a case in which the predetermined condition is detected, the identification data application unit may be configured to group, as the same group, one or more sets of image data that have been stored in the storage unit until the predetermined condition is detected and that have not been previously grouped, and to store, in the storage unit, a different item of the identification data for each group in association with the sets of image data.

In the first aspect, the identification data may be dummy data of the image data.

In the first aspect, the identification data application unit may be configured to store the dummy data of the image data at a position partitioning the sets of image data in the same group in a storage area of the storage unit.

In the first aspect, the identification data may be information that is different from subject information relating to a subject of which a radiographic image is acquired by the imaging unit.

In the first aspect, the radiographic imaging device may further include an operation unit that is configured to be operated by a user, and the predetermined condition may be a condition indicating that the operation unit has been operated.

In the first aspect, the radiographic imaging device may further include a detection unit that is configured to detect a predetermined state of the imaging device, wherein the predetermined condition is a condition indicating that the predetermined state has been detected by the detection unit.

In the first aspect, the detection unit may be a sensor detecting a movement state of the imaging device; and the predetermined state may be a state acquired by the sensor, indicating that the imaging device is moved by a predetermined distance or more.

In the first aspect, the detection unit may be a sensor that detects a change in a posture of the imaging device; and the predetermined state may be a state acquired by the sensor, in which the posture of the imaging device has changed from a first state to a second state.

In the first aspect, the detection unit may be a sensor that detects pressure applied to the imaging device; and the predetermined state may be a state acquired by the sensor, in which the pressure applied to the imaging device has changed by a predetermined amount or more.

In the first aspect, the detection unit may be a reception unit that receives a signal indicating an approach to at least one selected from the group consisting of a predetermined device and predetermined equipment; and the predetermined state is a state in which the reception unit has received the signal.

In the first aspect, the detection unit may be a sensor that detects an approach to at least one selected from the group consisting of a predetermined device and predetermined equipment; and the predetermined state may be a state indicating detection of an approach to the at least one selected from the group consisting of the predetermined device and the predetermined equipment.

In the first aspect, the detection unit may be a reception unit that receives a signal indicating a separation from at least one selected from the group consisting of a predetermined device and predetermined equipment; and the predetermined state may be a state in which the reception unit has received the signal.

In the first aspect, the detection unit may be a sensor that detects a separation from at least one selected from the group consisting of a predetermined device and predetermined equipment; and the predetermined state may be a state indicating detection of a separation from the at least one selected from the group consisting of the predetermined device and the predetermined equipment.

In the first aspect, the radiographic imaging device may further include a power source unit to which source power is externally supplied, and the predetermined condition may be a condition corresponding to source power externally supplied to the power source unit.

In the first aspect, the predetermined condition may be a condition corresponding to a position between the radiation irradiation device that irradiates the subject with radiation and at least one selected from the group consisting of a predetermined device and predetermined equipment.

A second aspect of the present disclosure is a radiographic imaging system including: the radiographic imaging device according to the first aspect; and a control device that includes: a reading unit that is configured to read the image data and the identification data stored in the storage unit of the radiographic imaging device; and a control unit that is configured to effect control of storing the image data and subject information relating to a subject corresponding to the image data in an image storing unit in association with each other based on the identification data.

In the second aspect, the control device may further include: a display unit that displays one or more sets of image data of a group partitioned by the identification data so as to be grouped for each group; and a reception unit that receives the subject information relating to the subject corresponding to the image data displayed on the display unit, and the control unit may be configured to effect control of storing the subject information received by the reception unit in the image storing unit in association with the corresponding image data.

In the second aspect, the control unit may be configured to acquire the subject information from order information of the radiographic image corresponding to the image data partitioned by the identification data, and to effect control of storing the acquired subject information in the image storing unit in association with the image data.

A third aspect of the present disclosure is an identification information application method causing a computer to execute a process including: acquiring a radiographic image in accordance with irradiated radiation by using an imaging unit; storing sets of image data of plural radiographic images acquired by the imaging unit in a storage unit; and storing identification data in the storage unit in a case in which a predetermined condition is detected, the identification data being used for partitioning, between before and after the detection of the predetermined condition, the sets of image data stored in the storage unit.

A fourth aspect of the present disclosure is a non-transitory storage medium storing a program that causes a computer to execute an identification data application process, the identification data application process including: acquiring a radiographic image in accordance with irradiated radiation by using an imaging unit; storing sets of image data of plural radiographic images acquired by the imaging unit in a storage unit; and storing identification data in the storage unit in a case in which a predetermined condition is detected, the identification data being used for partitioning, between before and after the detection of the predetermined condition, the sets of image data stored in the storage unit.

According to the present disclosure, a subject and image data of a corresponding radiographic image can be easily associated with each other.

### BRIEF DESCRIPTION OF THE DRAWINGS

Exemplary embodiments will be described in detail based on the following figures, wherein:

FIG. 1 is a schematic configuration diagram that illustrates an example of a radiographic imaging system according to a first embodiment;

FIG. 2 is a block diagram that illustrates an example of the schematic configuration of a radiographic imaging device, a mobile information terminal device, and a console according to the first embodiment;

FIG. 3 is a flowchart that illustrates an example of the flow of an imaging process executed by an imaging control unit of the radiographic imaging device according to the first embodiment;

FIG. 4 is a flowchart that illustrates an example of the flow of identification information (data) application processing executed by the imaging control unit of the radiographic imaging device according to the first embodiment;

FIG. 5 is a schematic diagram that illustrates a specific example of image data and dummy data stored in a storage unit of the radiographic imaging device according to the first embodiment;

FIG. 6 is a flowchart that illustrates an example of the flow of an association processing executed by a control unit of the console according to the first embodiment;

FIG. 7 is a schematic diagram that illustrates a specific example of a state in which image data and subject information (data) are stored in a storage unit in association with each other in the specific example illustrated in FIG. 5;

FIG. 8 is a flowchart that illustrates an example of the flow of identification data application processing executed by an imaging control unit of a radiographic imaging device according to a second embodiment;

FIG. 9 is a schematic diagram that illustrates a specific example of image data and identification data stored in the storage unit;

FIG. 10 is a block diagram that illustrates an example of the schematic configuration of a radiographic imaging device, a mobile information terminal device, and a console according to a third embodiment;

FIG. 11 is a flowchart that illustrates an example of the flow of imaging processing executed by an imaging control unit of a radiographic imaging device according to Example 3-1;

FIG. 12 is a flowchart that illustrates an example of the flow of imaging processing executed by an imaging control unit of a radiographic imaging device according to Example 3-2;

FIG. 13 is a flowchart that illustrates an example of the flow of imaging processing executed by an imaging control unit of a radiographic imaging device according to Example 3-3;

FIG. 14 is a block diagram that illustrates an example of the schematic configuration of a radiographic imaging device, a mobile information terminal device, a console, and a housing device according to a fourth embodiment;

FIG. 15 is a flowchart that illustrates an example of the flow of imaging processing executed by an imaging control unit of the radiographic imaging device according to the fourth embodiment;

FIG. 16 is a block diagram that illustrates an example of the schematic configuration of a radiographic imaging device, a mobile information terminal device, a console, and a housing device according to a fifth embodiment;

FIG. 17 is a flowchart that illustrates an example of the flow of imaging processing executed by an imaging control unit of the radiographic imaging device according to the fifth embodiment;

FIG. 18 is a block diagram that illustrates an example of the schematic configuration of a radiation irradiation device, a radiographic imaging device, a mobile information terminal device, and a console according to a sixth embodiment;

FIG. 19 is a flowchart that illustrates an example of the flow of imaging processing executed by an imaging control unit of the radiographic imaging device according to the sixth embodiment;

FIG. 20 is a flowchart that illustrates an example of the flow of ID reading processing executed by an irradiation control unit of the radiation irradiation device according to the sixth embodiment;

FIG. 21 is a block diagram that illustrates an example of the schematic configuration of a radiographic imaging device, a mobile information terminal device, and a console according to a variation of the sixth embodiment;

FIG. 22 is a block diagram that illustrates an example of the schematic configuration of a radiation irradiation device, a radiographic imaging device, a mobile information terminal device, and a console according to a seventh embodiment;

FIG. 23 is a configuration diagram that illustrates an example of a casing and a notification unit of the radiation irradiation device according to the seventh embodiment;

FIG. 24 is a flowchart that illustrates an example of the flow of imaging processing executed by an imaging control unit of the radiographic imaging device according to the seventh embodiment;

FIG. 25 is a configuration diagram that illustrates an example of a radiation irradiation device as a visiting car according to the seventh embodiment;

FIG. 26 is a flowchart that illustrates an example of the flow of association processing executed by the console in a case in which the console automatically executes association; and

FIG. 27 is a block diagram that illustrates an example of the schematic configuration of a radiographic imaging device, a mobile information terminal device, a console, and a housing device of a case in which the first and third embodiments are combined.

### DETAILED DESCRIPTION

Hereinafter, embodiments according to the present disclosure will be described with reference to the drawings. In the drawings, the same reference numeral is appended to parts having the same function, and duplicated description thereof will be omitted.

### First Embodiment

In the present embodiment, a case will be described in which identification information (data) is assigned in a case in which an operation button provided in a radiographic imaging device is operated by a user such as a radiologist after a radiographic image is imaged by the radiographic imaging device.

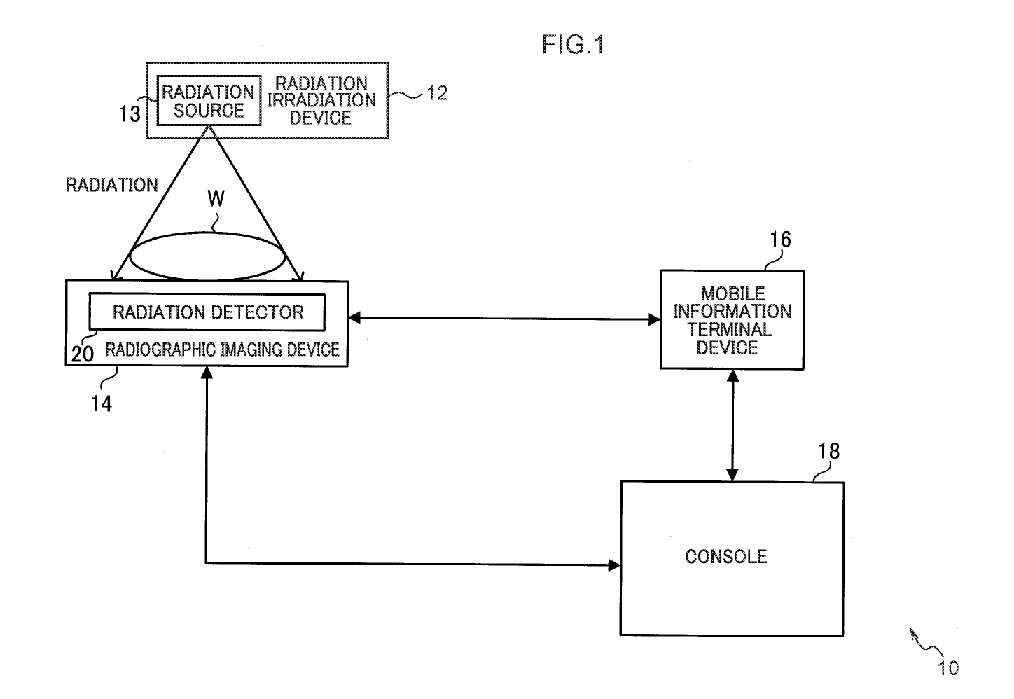

First, a schematic configuration of the radiographic imaging system according to the present embodiment will be described. FIG. 1 is a schematic configuration diagram that illustrates an example of the radiographic imaging system according to the present embodiment.

The radiographic imaging system 10 includes a radiation irradiation device 12, a radiographic imaging device 14, a mobile information terminal device 16, and a console 18.

The radiation irradiation device 12 includes a radiation source 13. The radiation irradiation device 12 emits radiation R (for example, X rays or the like) from the radiation source 13 to a subject W. Specific examples of the radiation irradiation device 12 include a visiting car and the like. The method of instructing the radiation irradiation device 12 to emit radiation R is not particularly limited. For example, the instruction method may be providing an irradiation button and the like in the radiation irradiation device 12, and giving an instruction for executing irradiation according to a user's operation of the irradiation button or the like. Alternatively, the instruction method may be, for example, giving by a user an instruction for execution of irradiation from an external device such as the mobile information terminal device 16 or the console 18.

The radiographic imaging device 14 includes a radiation detector 20 that is an example of an imaging unit according to the present disclosure, and that detects radiation R that has been emitted from the radiation irradiation device 12 and has been transmitted through the subject W (details are described later). The radiographic imaging device 14 acquires a radiographic image of the subject W by using the radiation detector 20. In the present embodiment, an electronic cassette is used as the radiographic imaging device 14.

The radiographic imaging device 14 generates and stores identification data in a case in which a predetermined condition is detected. In this embodiment, the “identification information (data)” is information used for partitioning image data stored in the storage unit 24 between before and after detection of the predetermined condition in a case in which the predetermined condition is detected, and does not include subject information (data) relating to the subject W.

The mobile information terminal device 16 according to the present embodiment serves as a part of the console 18 (for example, performs a function of controlling imaging of a radiographic image by the radiographic imaging device 14). The mobile information terminal device 16 according to the embodiment can be driven by a built-in battery and is a personal digital assistants (PDA). Specific examples of the mobile information terminal device 16 include a tablet terminal device, a smartphone, and the like.

The console 18, for example, controls the overall operation of the radiographic imaging system 10 and controls imaging of a radiographic image by the radiographic imaging device 14 based on instruction information (imaging menu information) input from an external system such as a radiology information system (RIS). For this reason, the console 18 receives order information including the imaging menu information from the external system.

In a case in which a radiographic image is acquired at a predetermined imaging location such as a ward, a hospital room, or an operating room, in the radiographic imaging system 10 according to the present embodiment, a radiographic image is acquired by arranging the radiation irradiation device 12, the radiographic imaging device 14, and the mobile information terminal device 16 at the target imaging location. In such a case, the imaging may be performed in a state in which the console 18 is arranged at a location (for example, an examination room of the department of radiology) different from the imaging location at which the radiation irradiation device 12, the radiographic imaging device 14, and the mobile information terminal device 16 are arranged.

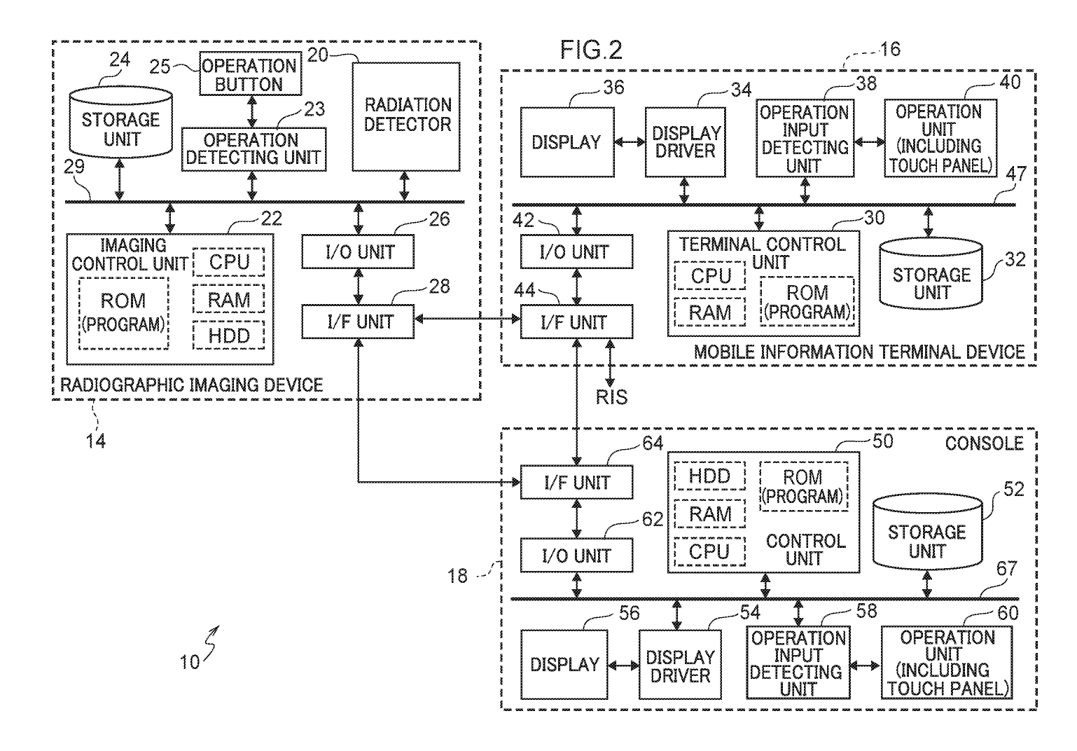

Next, the functions of the radiographic imaging device 14, the mobile information terminal device 16, and the console 18 will be described in detail. FIG. 2 is a block diagram that illustrates an example of the schematic configuration of the radiographic imaging device 14, the mobile information terminal device 16, and the console 18 of the radiographic imaging system 10.

The radiographic imaging device 14 according to the present embodiment includes a radiation detector 20, an imaging control unit 22, an operation detecting unit 23, a storage unit 24, an operation button 25, an I/O (input output) unit 26, and an I/F (interface) unit 28. The radiation detector 20, the imaging control unit 22, the operation detecting unit 23, the storage unit 24, and the I/O unit 26 are interconnected through a bus 29 such as a system bus or a control bus so as to be able to transmit and receive various kinds of information.

The radiographic imaging device 14 according to the present embodiment is not particularly limited, as long as it has a function of capturing a radiographic image according to radiation R transmitted through the subject W. For example, the radiographic imaging device 14 may be configured to acquire a radiographic image by detecting the start of irradiation of radiation R by itself asynchronously with the radiation irradiation device 12.

The radiation detector 20 detects radiations R transmitted through the subject W under the control of the imaging control unit 22. The radiation detector 20 according to the present embodiment is not particularly limited but may be a radiation detector of an indirect conversion type that converts radiation R into light and converts the converted light into electric charge, or a radiation detector of a direct conversion type that directly converts radiation R into electric charge.

The imaging control unit 22 controls the operation of the radiographic imaging device 14. Here, the imaging control unit 22 is an example of an identification data application unit according to the present disclosure and generates identification data and stores the generated identification data in the storage unit 24.

The imaging control unit 22 includes a central processing unit (CPU), a read only memory (ROM), a random access memory (RAM), and a hard disk drive (HDD). The CPU controls the overall operation of the radiographic imaging device 14. In the ROM, various processing program including an imaging processing program that is an example of an identification data application program according to the present disclosure executed by the CPU and the like are stored in advance. The RAM temporarily stores various kinds of data. The HDD stores and maintains various kinds of data. Here, the HDD may be a solid state drive (SSD) and may also be used as the storage unit 24.

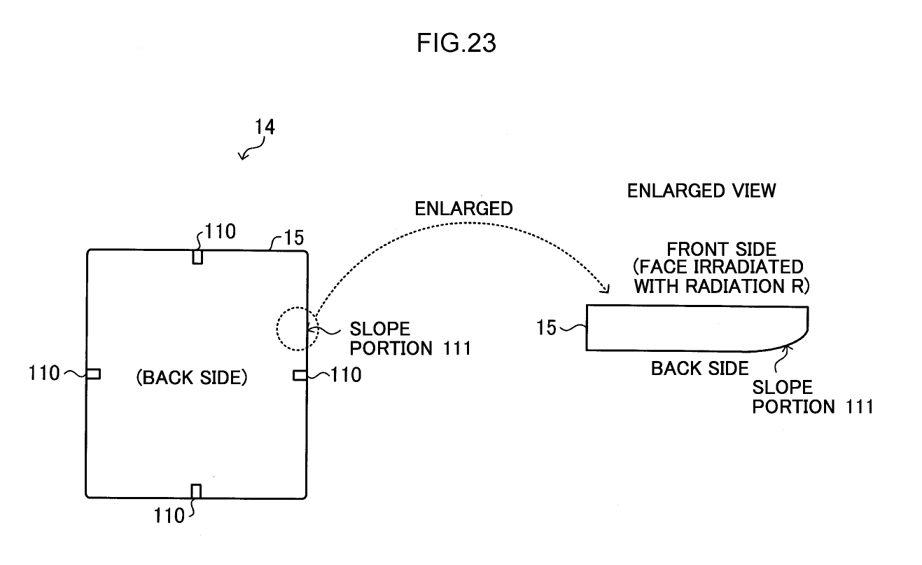

The operation button 25 that is an example of an operation unit according to the present disclosure is disposed at a casing (see a casing 15 of FIG. 23) of the radiographic imaging device 14, at a position not disturbing the imaging of a radiographic image. In the radiographic imaging device 14 according to the embodiment, the operation button 25 may be operated by the user after imaging of a series of radiographic images for the same subject W. The operation detecting unit 23 detects an operation of the operation button 25.

Image data, identification data, and the like of a radiographic image are stored in the storage unit 24 that is an example of a storage unit according to the present disclosure. A specific example of the storage unit 24 includes an SSD. The storage unit 24 may be detachable to the radiographic imaging device 14, and may be a universal serial bus (USB) memory, a secure digital (SD) Memory Card®, or the like.

The I/O unit 26 and the I/F unit 28 performs communication of various kinds of information (data) between the mobile information terminal device 16 and the console 18 through radio communication using electric waves, light or the like.

The mobile information terminal device 16 according to the embodiment includes a terminal control unit 30, a storage unit 32, a display driver 34, a display 36, an operation input detecting unit 38, an operation unit 40, an I/O unit 42, and an I/F unit 44. The terminal control unit 30, the storage unit 32, the display driver 34, the operation input detecting unit 38, and the I/O unit 42 are interconnected through a bus 47 such as a system bus or a control bus so as to be able to transmit and receive various kinds of information.

The terminal control unit 30 controls the overall operation of the mobile information terminal device 16. In a case in which order information of imaging is acquired from an external device such as the console 18, the terminal control unit 30 acquires the order information through the I/F unit 44.

The terminal control unit 30 includes a CPU, a ROM, and a RAM. The CPU controls the overall operation of the mobile information terminal device 16. In the ROM, various processing programs executed by the CPU and the like are stored in advance. The RAM temporarily stores various kinds of data.

The display 36 according to the embodiment displays various kinds of information such as information relating to the radiographic imaging device 14. The display driver 34 controls display of the various kinds of information on the display 36.

The operation unit 40 is used by a user for inputting an instruction, various kinds of information and the like relating to imaging of a radiographic image. In the present embodiment, the operation unit 40, for example, includes a touch panel, a touch pen, keys, a mouse, and the like. In a case in which the operation unit 40 is configured by a touch panel, the operation unit 40 may be integrated with the display 36. The operation input detecting unit 38 detects an operation state for the operation unit 40.

The I/O unit 42 and the I/F unit 44 communicate various kinds of information with the radiographic imaging device 14 and the console 18 through radio communication using electric waves or light.

In the storage unit 32, the order information described above and the like are stored. A specific example of the storage unit 32 includes an SSD.

The console 18 according to the embodiment, which is an example of a control device of to the present disclosure, is a server computer. The console 18 includes a control unit 50, a storage unit 52, a display driver 54, a display 56, an operation input detecting unit 58, an operation unit 60, an I/O unit 62, and an I/F unit 64. The control unit 50, the storage unit 52, the display driver 54, the operation input detecting unit 58, and the I/O unit 62 are interconnected through a bus 67 such as a system bus or a control bus so as to be able to transmit and receive various kinds of information.

The control unit 50, which is an example of a control unit of a control device according to the present disclosure, controls the overall operation of the console 18 and includes a CPU, a ROM, a RAM, and an HDD. The CPU controls the overall operation of the console 18. In the ROM, various processing programs executed by the CPU and the like are stored in advance. The RAM temporarily stores various kinds of data. The HDD stores and maintains various kinds of data.

The display 56 according to the embodiment, which is an example of a display unit of the control device according to the present disclosure, displays various kinds of information such as a radiographic image of image data. The display driver 54 controls the display of the various kinds of information on the display 56. The operation unit 60 is used by a user for inputting information relating to imaging of a radiographic image, subject information, and the like. In the present embodiment, the operation unit 60, for example, includes a touch panel, a touch pen, keys, a mouse, and the like. In a case in which the operation unit 60 is configured by a touch panel, the operation unit 60 may be integrated with the display 56. The operation input detecting unit 58, which is an example of a reception unit of a control device according to the present disclosure, detects an operation state for the operation unit 60.

The I/O unit 62 and the I/F unit 64 communicates various kinds of information with the radiographic imaging device 14 and the mobile information terminal device 16 through radio communication using electric waves or light.

In the storage unit 52, which is an example of an image storing unit according to the present disclosure, image data of a radiographic image and subject information (data) are stored in association with each other. Specific examples of the storage unit 52 include an HDD, an SSD, and the like.

The operation of the radiographic imaging system 10 according to the present embodiment will be described in which a radiographic image is imaged. In the present embodiment, a case will be described in which identification data is stored in the storage unit 24 in a case in which a user's operation for the operation button 25 is detected.

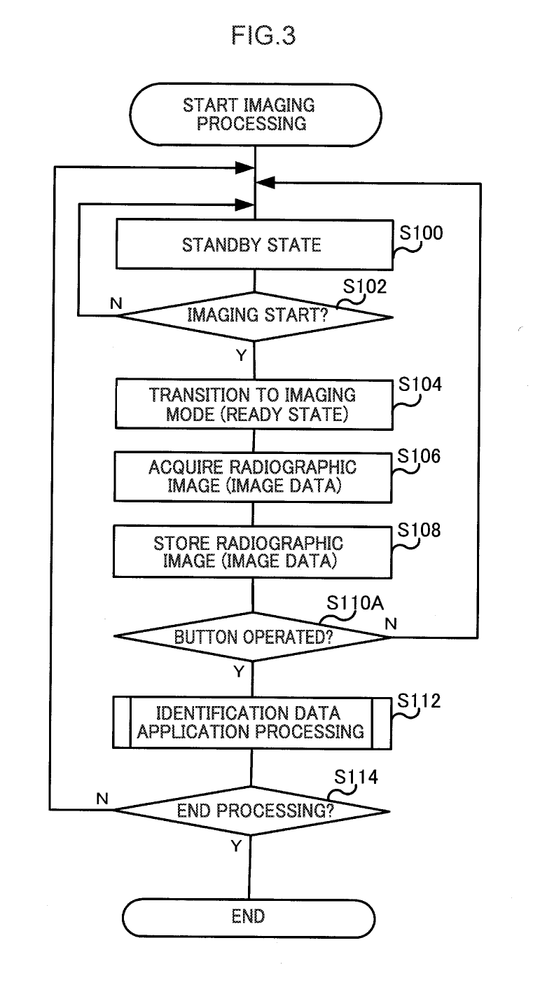

FIG. 3 is a flowchart that illustrates an example of the flow of imaging processing executed by the imaging control unit 22 of the radiographic imaging device 14 according to the embodiment. In the radiographic imaging device 14 according to the present embodiment, the imaging process is executed by the imaging control unit 22 executing an imaging processing program stored in its own ROM.

The imaging processing is executed after a power switch (not illustrated in the drawing) of the radiographic imaging device 14 is turned on.

In Step S100 illustrated in FIG. 3, the imaging control unit 22 causes the radiation detector 20 to be in a standby state. In the present embodiment, the “standby state” represents a state in which the power of the radiographic imaging device 14 is On, and the radiographic imaging device 14 is waiting for an instruction for imaging a radiographic image.

In the next Step S102, the imaging control unit 22 determines whether or not to start imaging of a radiographic image. In the radiographic imaging system 10 according to the embodiment, the mobile information terminal device 16 instructs the radiographic imaging device 14 to image a radiographic image.

In a case in which order information relating to the imaging is acquired from the console 18, the mobile information terminal device 16 instructs the radiographic imaging device 14 to image a radiographic image according to the acquired order information. In a case in which order information relating to the imaging is not acquired, the mobile information terminal device 16 instructs the radiographic imaging device 14 to perform imaging according to a setting that is set by a user's instruction operation.

In a case in which an instruction for imaging a radiographic image is given from the mobile information terminal device 16, in Step S102, the imaging control unit 22 determines to start imaging of a radiographic image. In a case in which it is determined, by the imaging control unit 22 in Step 102, not to start imaging of a radiographic image, the process returns to Step S100 and returns to a standby state. However, in a case in which it is determined to start imaging of a radiographic image by the imaging control unit 22, the process proceeds to Step S104. In Step S104, the imaging control unit 22 causes the radiation detector 20 to transit to an imaging mode that is a state in which detection of radiation R can be instantly performed, and causes the radiation detector 20 to be in a ready state. In the present embodiment, the “ready state” represents a state in which the radiation detector 20 can perform imaging and waiting for irradiation of radiation R. In the ready state, the radiation detector 20 may perform an operation for detecting irradiation of radiation R and the like and, therefore, the power consumption is higher than that in the standby state.

In a case in which the radiation irradiation device 12 is instructed to acquire a radiographic image from the mobile information terminal device 16, the radiation irradiation device 12 emits radiation R.

According to the above control processing executed by the imaging control unit 22, the radiation detector 20 detects radiation R that are emitted from the radiation irradiation device 12 and are transmitted through the subject W, to image a radiographic image of the subject W.

After a radiographic image is imaged by the radiation detector 20, in the next Step S106, the imaging control unit 22 acquires image data of the radiographic image from the radiation detector 20 and, in the next Step S108, the imaging control unit 22 stores the acquired image data of the radiographic image in the storage unit 24.

In the next Step S110A, the imaging control unit 22 determines whether or not the operation button 25 is operated. In a case in which an operation of the operation button 25 has not been detected by the operation detecting unit 23, the imaging control unit 22 determines that the operation button 25 has not been operated, and the process retunes to Step S100. For example, in a case in which imaging of the same subject W is continuously performed or in a case in which a moving image is acquired (plural still images are continuously acquired), the process returns to Step S100, and the processing of obtaining image data of an acquired radiographic image from the radiation detector 20 and storing the acquired image data in the storage unit 24 is repeatedly executed.

However, in a case in which an operation of the operation button 25 has been detected by the operation detecting unit 23, the imaging control unit 22 determines that the operation button 25 has been operated, and the process proceeds to Step S112.

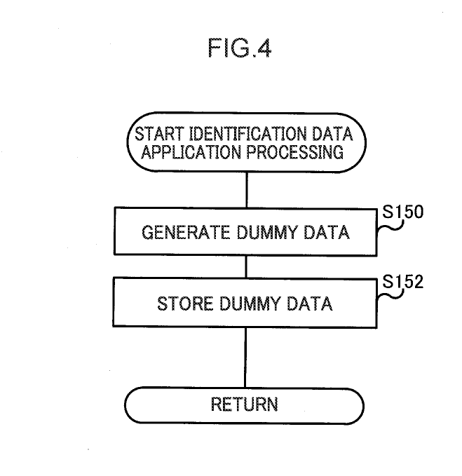

In Step S112, the imaging control unit 22 executes an identification data application processing. FIG. 4 is a flowchart that illustrates an example of the flow of the identification data application processing executed by the imaging control unit 22 according to the present embodiment. In the identification data application processing illustrated in FIG. 4, dummy data of the image data of the radiographic image is used as identification data.

In Step S150, the imaging control unit 22 generates dummy data having the same specifications (the format and size) as those of that image data of the radiographic image. An image represented by dummy data generated in this step is not particularly limited, but examples thereof include image data of a white image, a black image, an image representing a predetermined mark or the like.

The dummy data is not particularly limited unless it is data generated through a predetermined procedure. For example, the dummy data may be data of different specifications from the image data of the radiographic image, or may be data having a smaller size than the image data of the radiographic image.

In the next Step S152, the imaging control unit 22 stores the generated dummy data in the storage unit 24 and then the identification data application processing ends. The imaging control unit 22 stores the dummy data in a storage area immediately after the image data of the radiographic image that is stored in the storage unit 24 by the process of Step S108 described above. Through this step, the dummy data as the identification data is appended to the image data.

After the identification data application processing ends in this way, the process returns to Step S114 of the imaging processing (see FIG. 3), and the imaging control unit 22 determines whether or not to end the imaging processing. In a case in which the imaging process is not to be ended, the process returns to Step S100, and the imaging processing is repeated. For example, in a case in which a radiographic image of another subject W is to be imaged, the imaging processing is repeated due to the process returning to Step S100. In such a case, the image data of the radiographic image that is acquired next by the process of Step S106 is stored in a storage area immediately after the dummy data that has been previously stored in the storage unit 24 by the identification data application processing of Step S112.

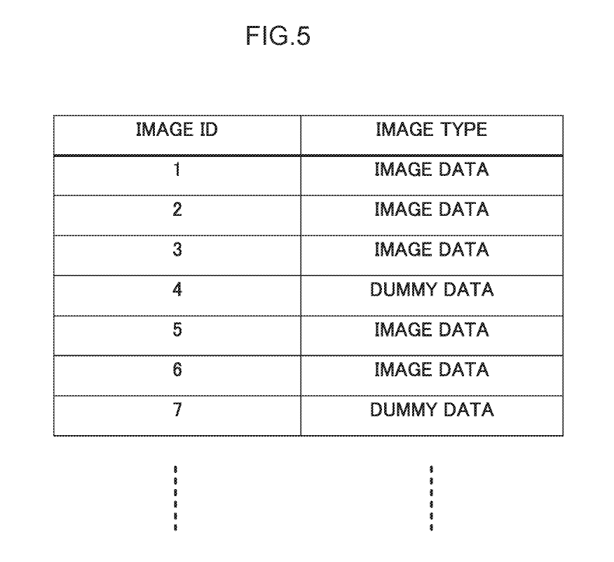

FIG. 5 is a schematic diagram that illustrates a specific example of image data and dummy data stored in the storage unit 24. The specific example of FIG. 5 illustrates a state in which image data of a series of radiographic images (for example, images of the same subject W) is stored as image data corresponding to image IDs (identification) 1 to 3, and dummy data generated by the imaging control unit 22 is stored as image data corresponding to an image ID 4. Further, the specific example of FIG. 5 illustrates a state in which image data of another series of radiographic images (for example, images of an subject W other than the same subject W described above) is stored as image data corresponding to image IDs 5 and 6, and dummy data is stored as image data corresponding to an image ID 7.

Meanwhile, in a case in which the power switch of the radiographic imaging device 14 is turned to an Off state or the like, affirmative determination is made in Step S114 by the imaging control unit 22, and the imaging processing ends.

In the imaging processing described above, a case has been described in which a standby state is formed after the start of the imaging process and the processing is not terminated after the identification data application processing. However, embodiments are not limited to this and, for example, a sleep state may be formed after at least one of the above conditions, instead of the standby state. Here, the “sleep state” is a state in which only the CPU of the imaging control unit 22 operates and is a state with power consumption lower than the standby state. In addition, a predetermined image ending process may be performed after the imaging of the radiographic image ends.

In the imaging processing described above, while the dummy data is applied after the timing of a radiographic image, the time at which the dummy data is applied may not be after the imaging processing. For example, the identification data application processing may be executed, dummy data may be generated, and the dummy data may be stored in the storage unit 24 in a case in which the operation button 25 is operated by the user before imaging a radiographic image.

In this way, in the radiographic imaging device 14 according to the present embodiment, in a case in which the operation button 25 is operated after the imaging of a radiographic image, the imaging control unit 22 generates dummy data as the identification data, and stores the generated dummy data after the image data of radiographic images acquired until the operation of the operation button 25, or before the image data of radiographic images to be acquired from now.

Thus, in the radiographic imaging device 14 according to the present embodiment, image data stored in the storage unit 24 is partitioned into before and after the detection of the predetermined condition by using the dummy data. This corresponds to all of the cases including: a case in which the dummy data is inserted between a series of image data and another series of image data; a case in which the dummy data is included at the end of a series of image data; and a case in which the dummy data is included at the start of a series of image data.

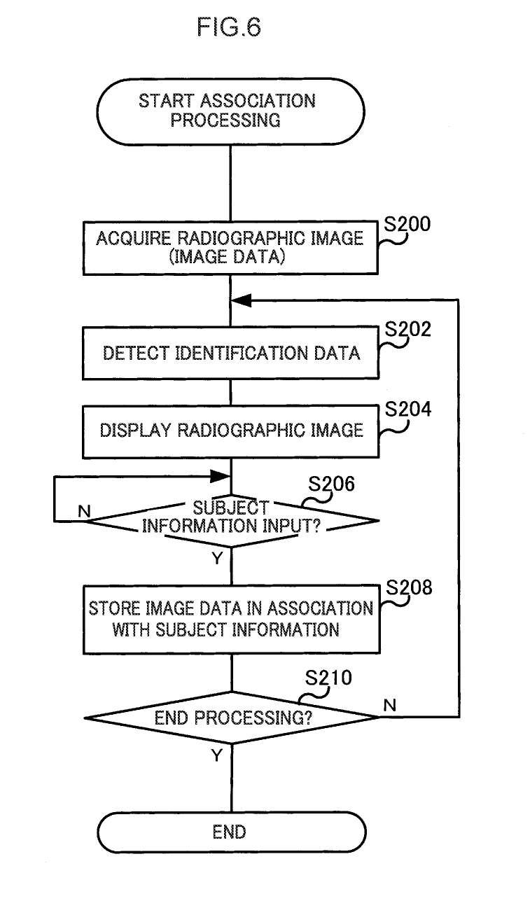

In the radiographic imaging system 10 according to the present embodiment, an association processing for associating image data of a radiographic image acquired by the radiographic imaging device 14 with subject information is executed by the control unit 50 of the console 18. FIG. 6 is a flowchart that illustrates an example of the flow of the association processing according to the present embodiment. This association processing is executed in a case in which an execution instruction is input through the operation unit 60.

In Step S200 illustrated in FIG. 6, the control unit 50 acquires image data of a radiographic image from the radiographic imaging device 14 through the I/F unit 64. In the present embodiment, as an example, all sets of image data of radiographic images that are stored in the storage unit 24 of the radiographic imaging device 14 are acquired, and the sets of image data are temporarily stored in the storage unit 52 in the same order as the stored order in the storage unit 24. The identification data (the dummy data in the present embodiment) is included in the image data of radiographic images acquired by the console 18 from the radiographic imaging device 14. Hereinafter, as a specific example, a case in which image data of radiographic images including the dummy data as illustrated in FIG. 5 is acquired will be described.

In Step S202, the control unit 50 detects dummy data from the acquired image data of radiographic images. The control unit 50 according to the embodiment detects each set of the dummy data in the stored order of the sets of the dummy data in the storage unit 52. For this reason, in the specific example, first, the dummy data corresponding to image ID 4 is detected.

In the next Step S204, the control unit 50 displays on the display 56 at least one radiographic image among the radiographic images represented by the series of image data partitioned by the detected dummy data. In the specific example described above, firstly, a radiographic image represented by at least one set of image data, among the sets of image data corresponding to image IDs 1 to 3 stored in the storage area before the dummy data corresponding to the image ID 4, is displayed on the display 56.

After checking the radiographic image displayed on the display 56, the user may input subject information relating to the subject W of the displayed radiographic image by using the operation unit 60. The subject information relating to the subject W is not particularly limited, and examples thereof include a subject ID, name, and the like. In a case in which the order information relating to the displayed radiographic image is acquired by the console 18, the order information may also be displayed together with the radiographic image.

In this way, according to the present embodiment, since the subject information is input by the user, in the next Step S206, the control unit 50 of the console 18 determines whether or not the subject information has been input. A standby state is maintained until the subject information is input, and, in a case in which the subject information is input, the process proceeds to Step S208.

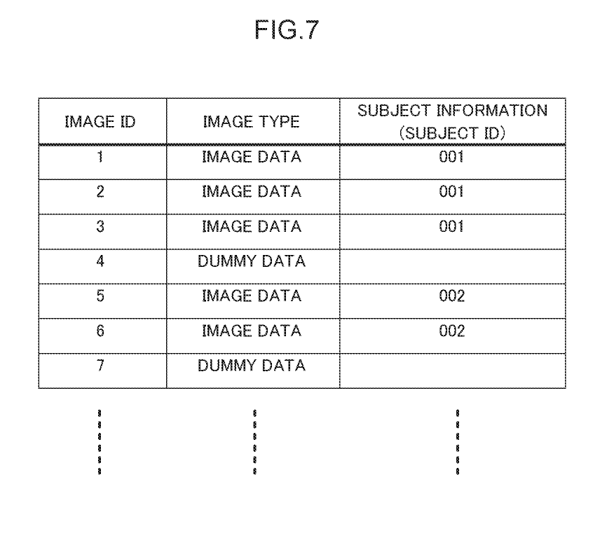

In Step S208, the control unit 50 stores the subject information input through the operation unit 60 in the storage unit 52 in association with the image data of the radiographic image displayed on the display 56. FIG. 7 is a schematic diagram that illustrates a specific example of a state in which image data and subject information are stored in the storage unit 52 in association with each other in the specific example illustrated in FIG. 5. FIG. 7 illustrates an example of a case in which a subject ID that is applied in advance differently for each subject W is input by the user as the subject information. In the example illustrated in FIG. 7, a subject ID “001” input by the user is stored in association with the image data corresponding to the image IDs 1 to 3. In addition, a subject ID “002” input by the user is stored in association with the image data corresponding to the image IDs 5 and 6.

In the next Step S210, the control unit 50 determines whether or not to terminate the association processing. In a case in which there is a set of image data not yet associated with subject information among the sets of image data of the radiographic images acquired in Step S200, the determination is negative, the process returns to Step S202, and the association processing is repeated. In a case where all sets of the image data of the radiographic images and the subject information are associated with each other, the determination is affirmative and the association processing ends.

In this way, according to the console 18 of the present embodiment, the subject information and the image data of a radiographic image can be easily stored in association with each other. As a result, a subject W and image data of a corresponding radiographic image can be easily associated with each other.

### Second Embodiment

While a case has been described in which the dummy data is applied as the identification data in the first embodiment, in the present embodiment, a case will be described in which other information is applied as the identification data. In order to identify a group of each of a series of image data, the present embodiment applies a different identification name for each set of image data or each group as the identification data. Examples of the identification name include an identification symbol, an identification number, and a name represented by a combination thereof.

The configuration (see FIGS. 1 and 2) of the radiographic imaging system 10 according to the embodiment is similar to that of the first embodiment and, therefore, detailed description thereof will be omitted.

In the processing executed by the radiographic imaging device 14 according to the present embodiment, only the identification data application processing is difference from that of the first embodiment and, therefore, hereinafter, the identification data application processing according to the present embodiment will be described. The flow (see FIG. 3) of the processing other than the identification data application processing in the imaging processing executed by the radiographic imaging device 14 is the same as that of the first embodiment.

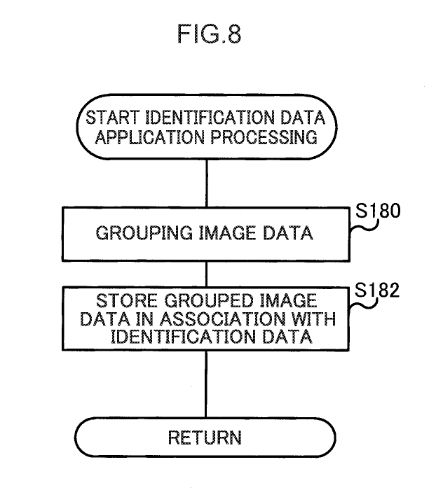

FIG. 8 is a flowchart that illustrates an example of the flow of the identification data application processing executed by the imaging control unit 22 of the radiographic imaging device 14 according to the present embodiment.

As illustrated in FIG. 8, in Step S180, the imaging control unit 22 specifies a set of image data not associated with an identification name among the sets of image data of the radiographic images stored in the storage unit 24, thereby grouping the sets of image data.

In the next Step S182, the imaging control unit 22 generates a new identification name and stores the generated identification name in the storage unit 24 in association with the image data specified by the process of Step S180 and then the identification data application processing ends.

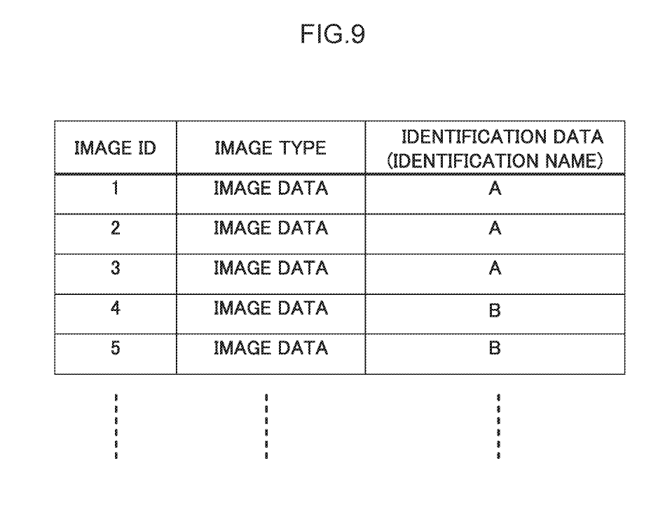

FIG. 9 is a schematic diagram that illustrates a specific example of image data and identification data stored the storage unit 24 by the imaging processing according to the present embodiment. The specific example illustrated in FIG. 9 shows a state in which the sets of image data corresponding to image IDs 1 to 3 and the sets of image data corresponding to image IDs 4 and 5 are respectively grouped, and the sets of image data are stored with mutually-different identification names A and B applied as identification data for the groups.

While a case in which an identification name generated for each group is stored in the storage unit 24 in association with each image data is illustrated in the specific example of FIG. 9, the method of applying identification names is not limited thereto. For example, information representing sets of image data included in each group may be generated as group information, and the generated group information and an identification name (identification data) may be stored in the storage unit 24 in association with each other.

In this way, in the radiographic imaging device 14 according to the present embodiment, in a case in which the operation button 25 is operated after imaging of a series of radiographic images has been executed for the same subject, the imaging control unit 22 groups the sets of image data of the series of radiographic images, and stores identification data in the storage unit 24 in association with the group or the sets of image data.

The overall processing of the association processing executed by the console 18 according to the present embodiment is similar to the association processing (see FIG. 6) according to the first embodiment. However, in the association processing of the present embodiment, instead of associating the image data and the subject information with each other, which is executed in Step S208, image data of a radiographic image and subject information are associated with each other by associating different subject information for each sets of image data to which common identification data is applied.

In this way, the console 18 of the present embodiment may easily store subject information and image data of a radiographic image in association with each other. As a result, the subject W and the image data of the corresponding radiographic image can be easily associated with each other.

### Third Embodiment

In the embodiments described above, a case has been illustrated in which the identification data is applied in a case in which an operation of the operation button 25 is detected. In the present embodiment, a case will be described in which the identification data is applied at different timing.

In a radiographic imaging device 14 according to the present embodiment, the identification data is applied to image data in a case in which its own device state becomes a predetermined state.

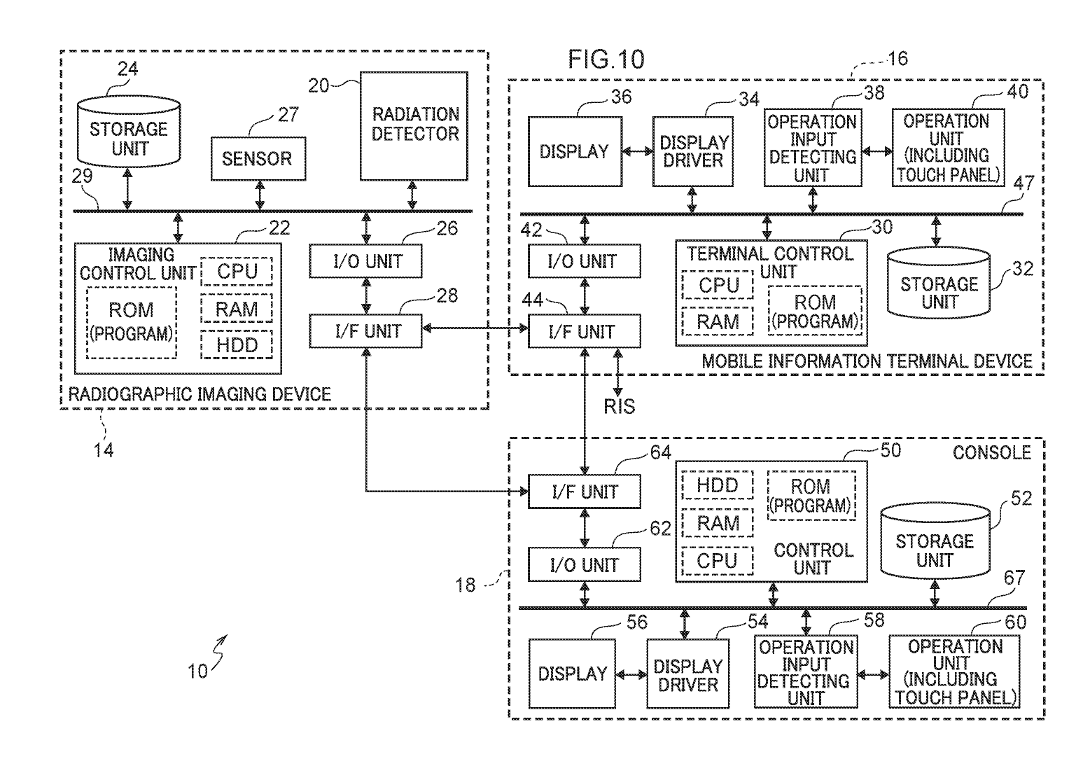

FIG. 10 is a block diagram that illustrates an example of the schematic configuration of a radiographic imaging device 14, a mobile information terminal device 16, and a console 18 according to the present embodiment.

The configuration of the radiographic imaging system 10 according to the present embodiment is the same as that of the radiographic imaging system 10 according to the first embodiment except for the inclusion of a sensor 27 serving as an example of a detection unit, in place of the operation detecting unit 23 and the operation button 25 included in the radiographic imaging device 14 according to the first embodiment.

The sensor 27 detects the state of the radiographic imaging device 14. The state of the radiographic imaging device 14 that is detected by the sensor 27 refers to a state except for the states of the radiographic imaging device 14 relating to imaging operation.

Specific examples of the state described above include a state of movement (for example, movement due to carriage) of the radiographic imaging device 14, a state of posture of the radiographic imaging device 14, and a state of pressure applied to the radiographic imaging device 14.

Hereinafter, the specific examples described above will be described in detail as examples of the present embodiment.

### Example 3-1

In this example, a case will be described in which the movement state of the radiographic imaging device 14 is applied as the state of the radiographic imaging device 14. The sensor 27 of such a case is not particularly limited, as long as it is a sensor detecting the movement of the radiographic imaging device 14. Specific examples of the sensor 27 include a velocity sensor, an acceleration sensor, and the like. Hereinafter, a case in which an acceleration sensor is applied as the sensor 27 will be described.

Generally, in order to acquire radiographic images, the radiographic imaging device 14 is moved to a location at which imaging processing is performed, and then the imaging processing is performed. After the imaging processing for the same subject W ends, the radiographic imaging device 14 may often be moved to a location at which the next imaging processing is performed or a location for housing the radiographic imaging device 14. For this reason, in the radiographic imaging device 14 of this example, the movement state of the radiographic imaging device 14 is regarded as a state for performing partitioning of image data of a series of radiographic images corresponding to the same subject W, and the identification data is applied to the image data.

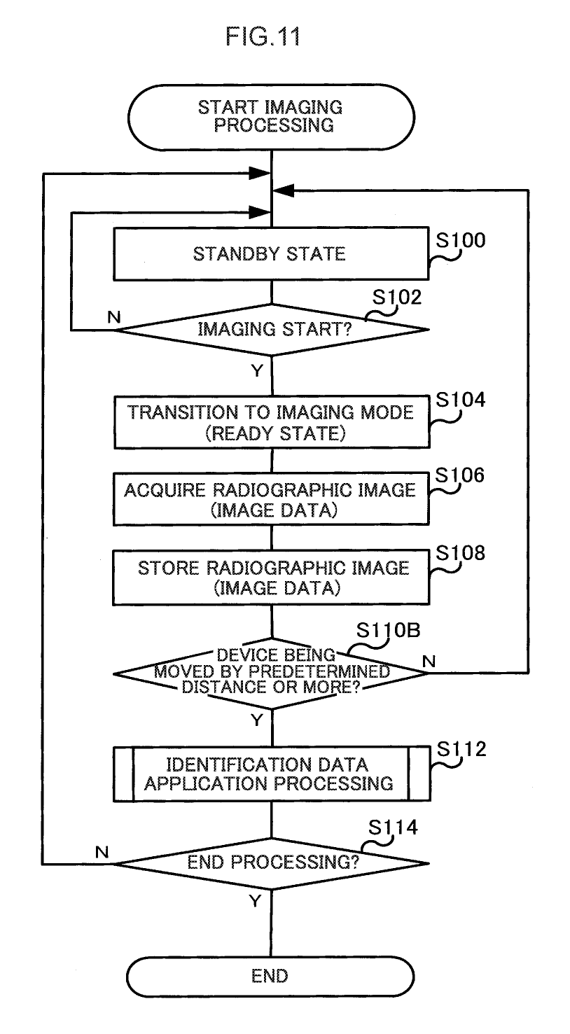

FIG. 11 is a flowchart that illustrates an example of the flow of the imaging processing executed by the imaging control unit 22 of the radiographic imaging device 14 of this example. In FIG. 11, the same step number is assigned to a step executing the same process as in the imaging processing illustrated in FIG. 3, and description thereof will be omitted.

As illustrated in FIG. 11, the imaging processing of this example is as same as that of the first embodiment except for Step S110B is performed instead of Step S110A of the imaging processing according to the first embodiment.

In Step S110B, the imaging control unit 22 determines whether or not the radiographic imaging device 14 has been moved by a predetermined distance or more. More specifically, a time from the acceleration detected by the sensor 27, which is an acceleration sensor, reaches a predetermined threshold or more to the acceleration becomes a predetermined threshold or less is measured as a time from the start to the end of the movement. In a case in which the measured time is a predetermined time or more, it is determined that the radiographic imaging device 14 has been moved by a predetermined distance or more. The predetermined distance corresponds to a distance of the movement from power of the radiographic imaging device 14 been turned on and until the radiographic imaging device 14 being arranged at an imaging location. The predetermined distance is set so as to exclude cases in which the radiographic imaging device 14 is moved after being transitioned to the imaging mode due to movement of the subject W, the positioning of the subject W, or the like during the imaging processing. The predetermined time may be determined in consideration of the imaging location, the positioning of the subject W, an imaging site, and the like.

In a case in which the determination is negative in Step S110B, the imaging control unit 22 returns the process to Step S100. In a case in which the determination is affirmative in Step S110B, the process proceeds to Step S112.

In this way, in the radiographic imaging device 14 of this example, in a case in which it is determined that the radiographic imaging device 14 has been moved by the predetermined distance or more based on a detection result acquired by the sensor 27, which is an acceleration sensor, the imaging control unit 22 stores image data of a radiographic image in the storage unit 24 with identification data being applied thereto.

### Example 3-2

In this example, a case in which the posture state of the radiographic imaging device 14 is applied as the state of the radiographic imaging device 14 will be described.

The posture of the radiographic imaging device 14 may be different depending on whether or not imaging of a radiographic image is performed. For example, when performing imaging processing, the imaging process may be performed in a state in which the radiographic imaging device 14 is horizontally placed (a state in which the radiographic image detection face faces upward) and the subject W is positioned on the radiographic imaging device 14. In such cases, when the imaging process ends and the radiographic imaging device 14 is moved, the radiographic imaging device 14 may be moved in an upright state (a state in which the radiographic image detection face faces the horizontal direction).

For this reason, in this example, the posture of an upright state and the posture of a horizontally arranged state thereof will be described as a specific example of the posture of the radiographic imaging device 14.

The sensor 27 of such a case is not particularly limited as long as it is a sensor that can detect a change in the facing direction (upward direction or horizontal direction) of the radiographic imaging device 14. Specific examples thereof include a gyro sensor, an acceleration sensor, and the like. Hereinafter, a case in which the gyro sensor is applied as the sensor 27 will be described.

The posture of the radiographic imaging device 14 is not limited to this example. For example, in a case in which a subject W lying on a bed with the upper body being inclined, instead of the upward direction, the radiographic imaging device 14 may be inclined by an angle according to the angle of the bed, a posture change between this angle and the horizontal direction may be detected by the sensor 27. The posture of the radiographic imaging device 14 may be any other posture, and, in such a case, a sensor that is suitable for detecting a change in the posture may be used.

Thus, in the radiographic imaging device 14 of this example, a state in which the posture of the radiographic imaging device 14 is changed is regarded as a state for partitioning image data of a series of radiographic images corresponding to the same subject W, and the identification data is applied to the image data.

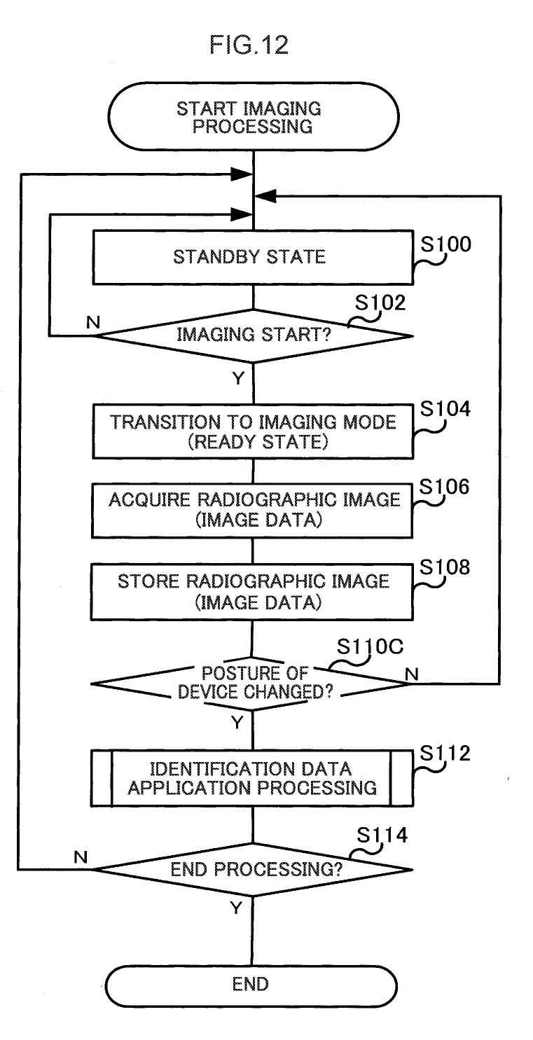

FIG. 12 is a flowchart that illustrates an example of the flow of the imaging processing executed by the imaging control unit 22 of the radiographic imaging device 14 of this example. In FIG. 12, the same step number is assigned to a step executing the same process as in the imaging processing illustrated in FIG. 3, and description thereof will be omitted.

As illustrated in FIG. 12, the imaging processing of this example is the same as that of the first embodiment except for Step S110C in place of Step S110A of the imaging processing according to the first embodiment.

In Step S110C, the imaging control unit 22 determines whether or not the posture of the radiographic imaging device 14 has been changed. More specifically, in a case in which the sensor 27, which is a gyro sensor, detects that the angle of the radiographic imaging device 14 have been changed by a predetermined threshold value or more, it is determined that the posture of the radiographic imaging device 14 have been changed.

In a case in which the determination is negative in Step S110C, the imaging control unit 22 causes the process to return to Step S100. In a case in which the determination is affirmative in Step S110C, the imaging control unit 22 causes the process to proceed to Step S112.

In this way, in a case in which it is determined that the posture of the radiographic imaging device 14 has been changed based on a detection result acquired by the sensor 27 that is the gyro sensor, the imaging control unit 22 of the radiographic imaging device 14 of this example stores image data of a radiographic image in the storage unit 24 with identification data being applied thereto.

### Example 3-3

In this example, a case in which the state of an external force applied to the radiographic imaging device 14 is applied as the state of the radiographic imaging device 14 will be described. While a case of considering a pressure as a specific example of the external force will be described in this example, the external force is not limited thereto and may be an external force due to any other impact.

The sensor 27 of the case in which a pressure is regarded as the external force is not particularly limited, as long as it is a sensor that can detect a change in a pressure applied to the radiographic imaging device 14. Specific examples of the sensor 27 include various pressure sensors such as a strain sensor and the like.

In a case in which imaging of a radiographic image is performed, pressure applied to the radiographic imaging device 14 may be higher than that of a case in which imaging is not performed, due to positioning of the subject W on the radiographic imaging device 14 or due to the subject W contacting the radiographic imaging device 14. In such cases, the pressure applied to the radiographic imaging device 14 changes between before and after the imaging.

For this reason, in the radiographic imaging device 14 of this example, a state in which a pressure applied to the radiographic imaging device 14 is changed by a predetermined amount or more is regarded as a state for partitioning image data of a series of radiographic images corresponding to the same subject W, and the identification data is applied to the image data.

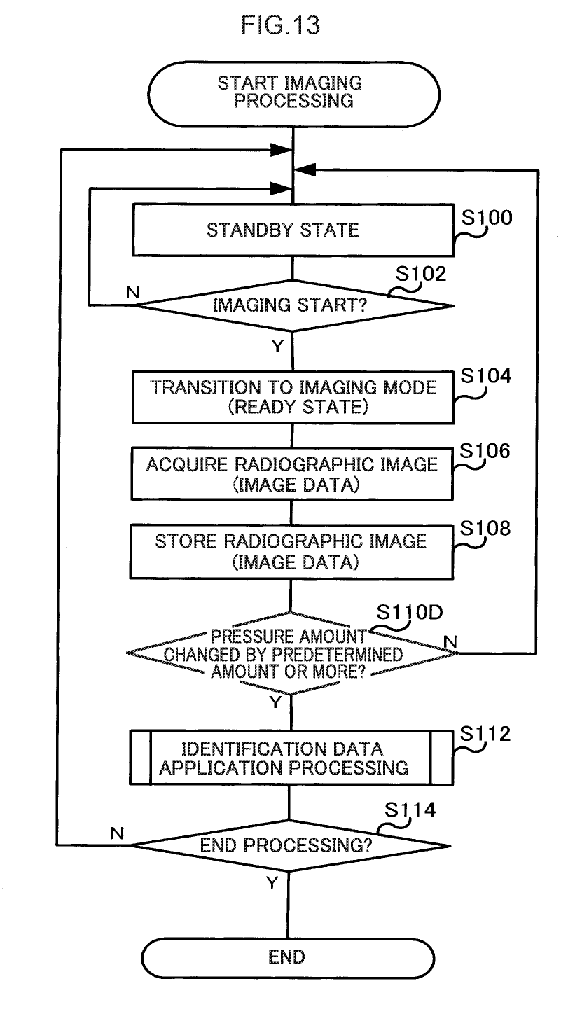

FIG. 13 is a flowchart that illustrates an example of the flow of the imaging processing executed by the imaging control unit 22 of the radiographic imaging device 14 of this example. In FIG. 13, the same step number is assigned to a step executing the same process as in the imaging process illustrated in FIG. 3, and description thereof will be omitted.

As illustrated in FIG. 13, the imaging processing of this example is the same as that of the first embodiment except for performing Step S110D in place of Step S110A of the imaging processing according to the first embodiment.

In Step S110D, the imaging control unit 22 determines whether or not the pressure applied to the radiographic imaging device 14 has been changed by a predetermined amount or more. More specifically, the sensor 27 that is a pressure sensor detects the pressure applied to the radiographic imaging device 14 and determines whether or not the detected pressure has been changed by the predetermined amount or more.

In a case in which the determination is negative in Step S110D, the imaging control unit 22 causes the process to return to Step S100. In a case in which the determination is affirmative in Step S110D, the imaging control unit 22 causes the process to proceed to Step S112.

In this way, in the radiographic imaging device 14 of this example, in a case in which it is determined that the pressure applied to the radiographic imaging device 14 has been changed by the predetermined amount or more based on the detection result acquired by the sensor 27 that is the pressure sensor, the imaging control unit 22 stores image data of a radiographic image in the storage unit 24 with the identification data being applied thereto.

In a case in which the external force is due to any other impact, for example, the impact amount is detected by using an acceleration sensor or the like, and the impact amount may be applied instead of the pressure amount in the imaging processing.

In this way, in a case in which it is determined that the radiographic imaging device 14 is in a predetermined state, the imaging control unit 22 of the radiographic imaging device 14 of this embodiment applies the identification data to image data of a radiographic image, and stores the identification data in the storage unit 24 in association with the image data.

The association processing executed by the console 18 according to this embodiment is similar to the association processing (see FIG. 6) of each of the embodiments described above, and thus, description thereof will be omitted.

In this way, according to the console 18 of this embodiment, similar to the console 18 of each embodiment described above, the subject information and the image data of a radiographic image can be easily stored in association with each other. As a result, the subject W and the image data of a corresponding radiographic image can be easily associated with each other.

The sensor 27 is not limited to that described above and may be any sensor that can detect any other state of the radiographic imaging device 14. For example, the sensor 27 may be a temperature sensor detecting heat applied to the radiographic imaging device 14. In a case in which imaging of a radiographic image is performed, heat applied to the radiographic imaging device 14 may be change due the subject W positioned on the radiographic imaging device 14, or contacting the radiographic imaging device 14. In such cases, it may be configured such that the temperature of the outer face of the casing of the radiographic imaging device 14 is detected, a state in which the detected temperature has been changed by a predetermined threshold value or more is regarded as the state for partitioning image data of a series of radiographic images corresponding to the same subject W, and the identification data is applied to the image data.

### Fourth Embodiment

In the third embodiment, a case in which the identification data is applied in a case in which the state of the radiographic imaging device 14 that is acquired by the sensor changes has been illustrated. In the present embodiment, a case will be described in which the identification data is applied in a case in which a signal representing an approach to a predetermined device is received the a radiographic imaging device 14.

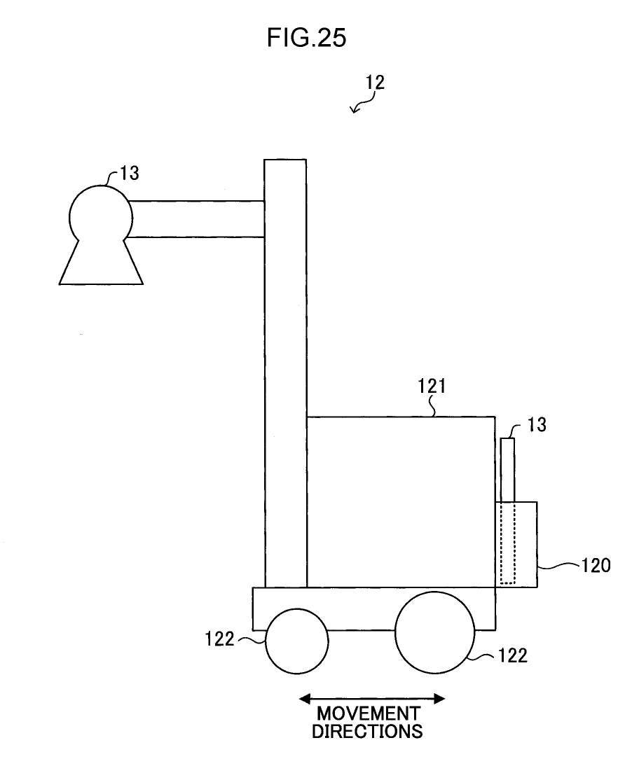

When imaging of a radiographic image is not performed, the radiographic imaging device 14 may be housed in a housing device for storage. Examples of such a housing device includes a storage case of a visiting car (a storage case 120 of FIG. 25), a housing device in which a charging function for charging a storage battery with power in case of the radiographic imaging device 14 including the storage battery.

In such a case, in order to perform imaging of a radiographic image, the radiographic imaging device 14 is taken out from the housing device and is arranged at an imaging location. Therefore, in the present embodiment, a state in which the radiographic imaging device 14 receives a signal representing an approach of the radiographic imaging device 14 to the housing device is regarded as a state for partitioning image data of a series of radiographic images corresponding to the same subject W, and the identification data is applied to the image data.

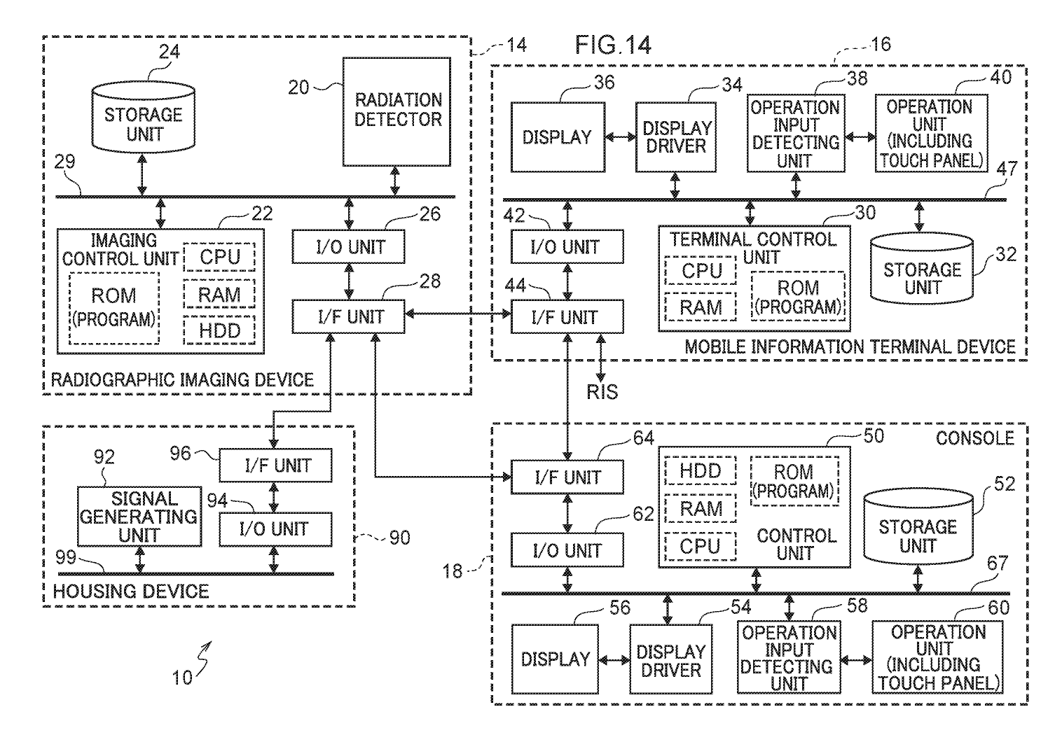

FIG. 14 is a block diagram that illustrates an example of the schematic configuration of a radiographic imaging device 14, a mobile information terminal device 16, a console 18, and a housing device 90 according to the present embodiment.

As illustrated in FIG. 14, the housing device 90 according to the present embodiment includes a signal generating unit 92, an I/O unit 94, and an I/F unit 96. The signal generating unit 92 and the I/O unit 94 are interconnected through a bus 99 such as a system bus or a control bus so as to be able to transmit and receive various kinds of information.

As illustrated in FIG. 14, the radiographic imaging device 14 according to the present embodiment is different from the radiographic imaging device 14 (see FIG. 2) according to the first embodiment only in that the operation detecting unit 23 and the operation button 25 are not provided.

The signal generating unit 92 of the housing device 90 generates a signal (hereinafter, referred to as an “approach signal”) used by the radiographic imaging device 14 to detect an approach of the radiographic imaging device 14 to the housing device 90. This approach signal is not particularly limited and, for example, may be a beacon of Bluetooth® or the like.

The radiographic imaging device 14 according to the present embodiment receives the approach signal transmitted from the housing device 90. Specifically, in the radiographic imaging device 14, the I/F unit 28 serves as an example of a reception unit and receives the approach signal transmitted from the housing device 90.

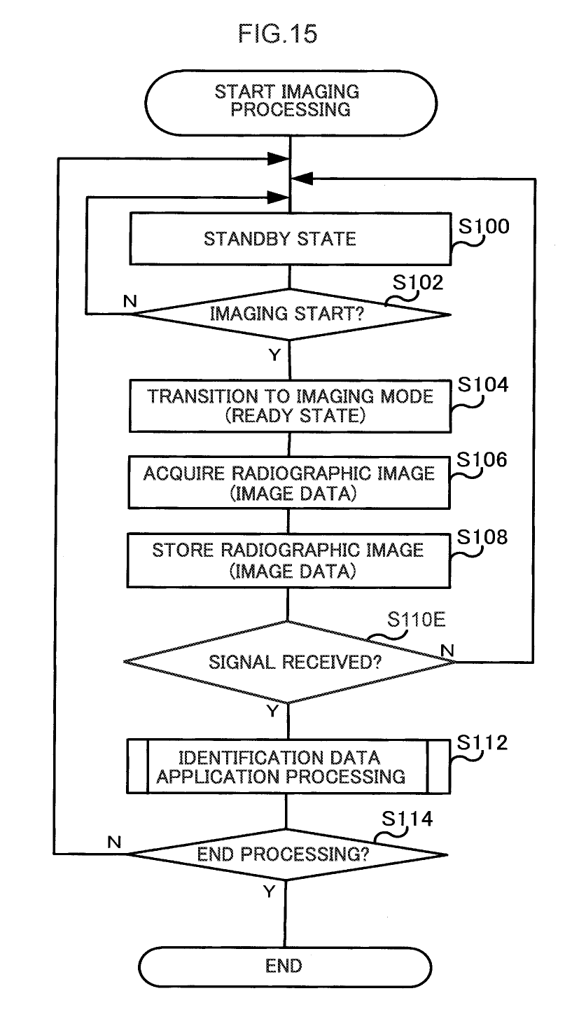

FIG. 15 is a flowchart that illustrates an example of the flow of an imaging processing executed by the imaging control unit 22 of the radiographic imaging device 14 according to the present embodiment. In FIG. 15, the same step number is assigned to a step executing the same process as in the imaging processing illustrated in FIG. 3, and description thereof will be omitted.

As illustrated in FIG. 15, the imaging processing of the present embodiment is the same as that of the first embodiment except for performing Step S110E in place of Step S110A of the imaging processing according to the first embodiment.

In Step S110E, the imaging control unit 22 determines whether or not an approach signal has been received from the housing device 90. The determination of whether or not the approach signal has been received may be performed by detecting the signal strength of the received signal, and determining that the approach signal has been received if the signal strength is a predetermined threshold value or more.

In a case in which the determination is negative in Step S110E, the imaging control unit 22 causes the process to return to Step S100. In a case in which the determination is affirmative, the imaging control unit 22 causes the process to proceed to Step S112.

In this way, in a case in which it is determined that an approach signal has been received from the housing device 90, the imaging control unit 22 of the radiographic imaging device 14 according to the present embodiment stores image data of a radiographic image in a storage unit 24 with identification data being applied thereto.

The association processing executed by the console 18 according to the present embodiment is similar to the association processing (see FIG. 6) of each of the embodiments described above, and thus, description thereof will be omitted.

In this way, according to the console 18 of the present embodiment, similar to the console 18 of each of the embodiments described above, the subject information and image data of a radiographic image can be easily stored in association with each other. As a result, the subject W and the image data of the corresponding radiographic image can be easily associated with each other.

### Fifth Embodiment

In the fourth embodiment described above, an example in which the identification data is applied in a case in which the radiographic imaging device 14 approaches the housing device, which is the predetermined device, has been illustrated. In the present embodiment, an example will be described in which the identification data is applied in accordance with the supply of source power to the radiographic imaging device 14.

As described in the third embodiment, in a case in which the radiographic imaging device 14 is stored in a housing device, the radiographic imaging device 14 may receive power supply from the housing device, and electric power may be accumulated (charged) in a power unit provided in the radiographic imaging device 14.

Therefore, in the radiographic imaging device 14 according to the present embodiment, a state in which power is supplied from the housing device to the radiographic imaging device 14 is regarded as a state for partitioning image data of a series of radiographic images corresponding to the same subject W, and the identification data is applied to the image data.

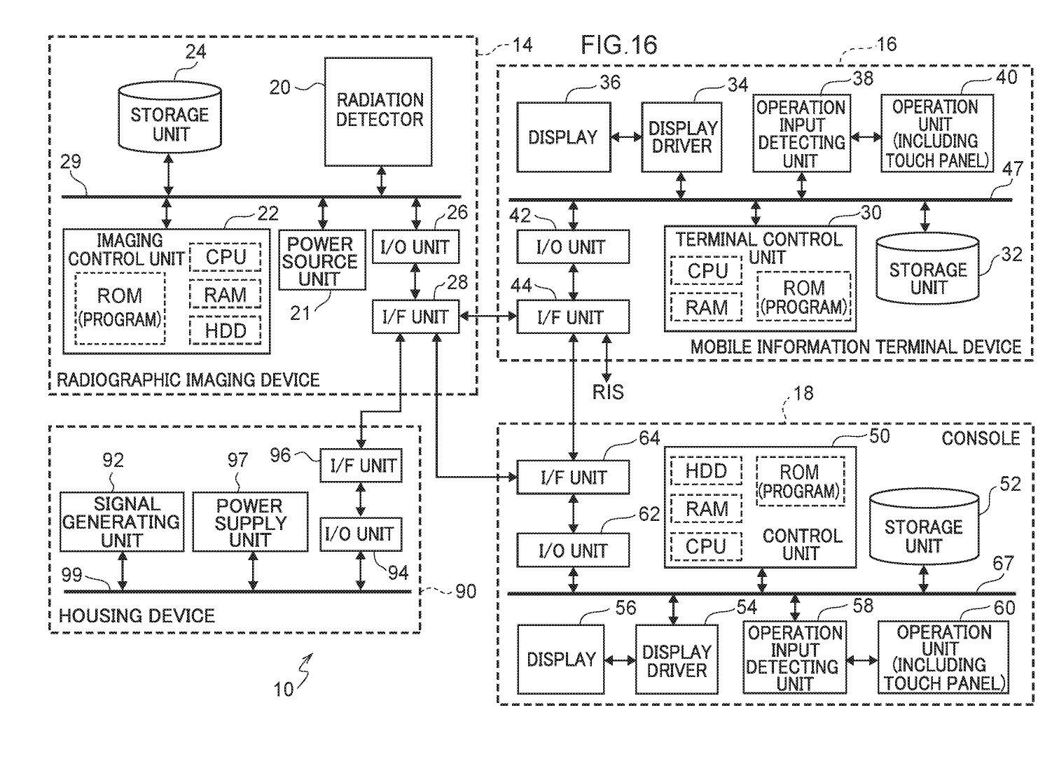

FIG. 16 is a block diagram that illustrates an example of the schematic configuration of a radiographic imaging device 14, a mobile information terminal device 16, a console 18, and a housing device 90 according to the present embodiment.

As illustrated in FIG. 16, the radiographic imaging device 14 according to the present embodiment is different from the radiographic imaging device 14 (see FIG. 14) according to the third embodiment in that it includes a power source unit 21. In addition, the housing device 90 is different from the housing device 90 (see FIG. 14) according to the third embodiment in that it includes a power supply unit 97. The power supply unit 97 supplies power to the power source unit 21 of the radiographic imaging device 14 that is housed in the housing device 90. The power source unit 21 of the radiographic imaging device 14 accumulates the supplied power, and the radiation detector 20 and the other units of the radiographic imaging device 14 are driven by the power accumulated in the power source unit 21.

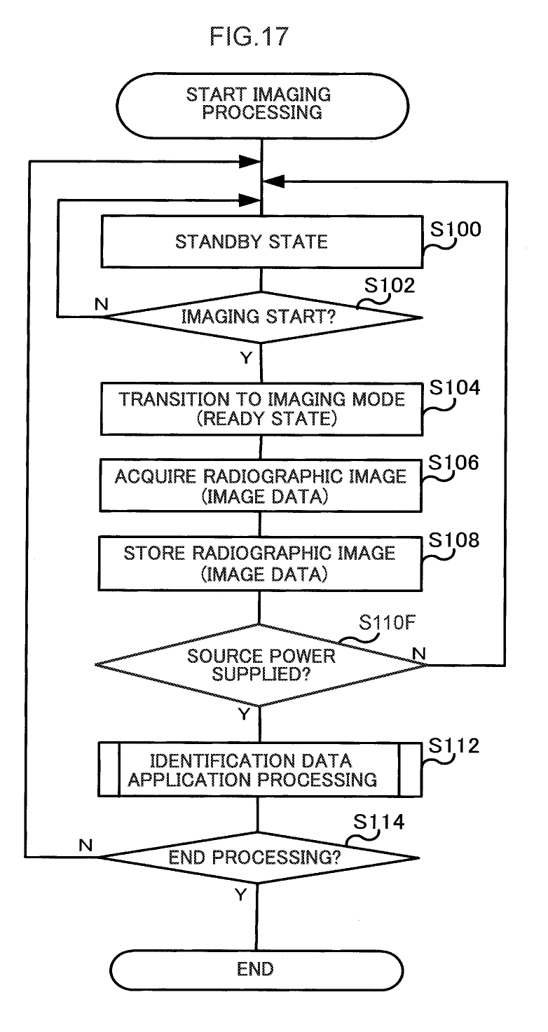

FIG. 17 is a flowchart that illustrates an example of the flow of an imaging processing executed by the imaging control unit 22 of the radiographic imaging device 14 according to the present embodiment. In FIG. 16, the same step number is assigned to a step executing the same process as in the imaging processing illustrated in FIG. 3, and description thereof will be omitted.

As illustrated in FIG. 16, the imaging processing according to the present embodiment is the same as that of the first embodiment except for performing Step S110F in place of Step S110A of the imaging processing according to the first embodiment.

In Step S110F, the imaging control unit 22 determines whether or not power is supplied from the power supply unit 97 of the housing device 90 to the power source unit 21. The determination of whether or not power is supplied may be performed by detecting the power charge amount of the power source unit 21, and, in a case in which the power storage amount increases, determining that the supply of source power is started and the power is supplied. The determination of whether or not the source power is supplied may not be made based on the start of supply of the source power, but may be made based on whether or not the supply of the source power has ended. In such a case, the power charge amount of the power source unit 21 may be determined, and, in a case in which an increase in the power charge amount stops, a determination may be made such that the supply of the source power has ended.

In a case in which the determination is negative in Step S110F, the imaging control unit 22 causes the process to return to Step S100. In a case in which the determination is affirmative in Step S110F, the imaging control unit 22 causes the process to proceed to Step S112.

In this way, in the radiographic imaging device 14 according to the present embodiment, in a case in which it is determined that the source power is supplied from the housing device 90, the imaging control unit 22 stores image data of a radiographic image in a storage unit 24 with the identification data being applied thereto.

The association processing executed by the console 18 according to the present embodiment is similar to the association processing (see FIG. 6) of the embodiments described above, and thus, description thereof will be omitted.

In this way, according to the console 18 of the present embodiment, similar to the console 18 of the embodiments described above, subject information and image data of a radiographic image can be easily stored in association with each other. As a result, the subject W and the image data of the corresponding radiographic image can be easily associated with each other.

### Sixth Embodiment

In the fourth embodiment described above, an example has been explained in which the identification data is applied in a case in which a signal has been received indicating that the radiographic imaging device 14 has approached the predetermined device. In the present embodiment, an example will be described in which the identification data is applied in a case in which a signal has been received indicating that the radiographic imaging device 14 has separated from predetermined equipment.

The radiographic imaging device 14 may be moved to another location after completion of imaging of radiographic images of a specific subject W. For example, after performing imaging of radiographic images of a particular subject W at a particular location, the radiographic imaging device 14 may be moved to another location where the next subject W is waiting. Specifically, after performing imaging of radiographic images for a particular subject W lying on a bed in a hospital room, imaging of radiographic images for the next subject W lying on a bed in the same or another hospital room may be performed.

Therefore, in the present embodiment, a state in which the radiographic imaging device 14 has received a signal indicating a separation of the radiographic imaging device 14 from the bed of the subject W is regarded as a state for partitioning image data of a series of radiographic images corresponding to the same subject W, and the identification data is applied to the image data.

In the present embodiment, a specific case will be described in which the radiation irradiation device 12 is a visiting car. In such a case, the radiation irradiation device 12 is also moved to a position corresponding to a bed of a subject W to be imaged, together with the radiographic imaging device 14.

In the present embodiment, the radiation irradiation device 12, which is a visiting car, reads information identifying the bed of the subject W to be imaged and transmits the information to the radiographic imaging device 14. The radiographic imaging device 14 receives the information identifying the bed of the subject W as a signal indicating a separation from a predetermined equipment.

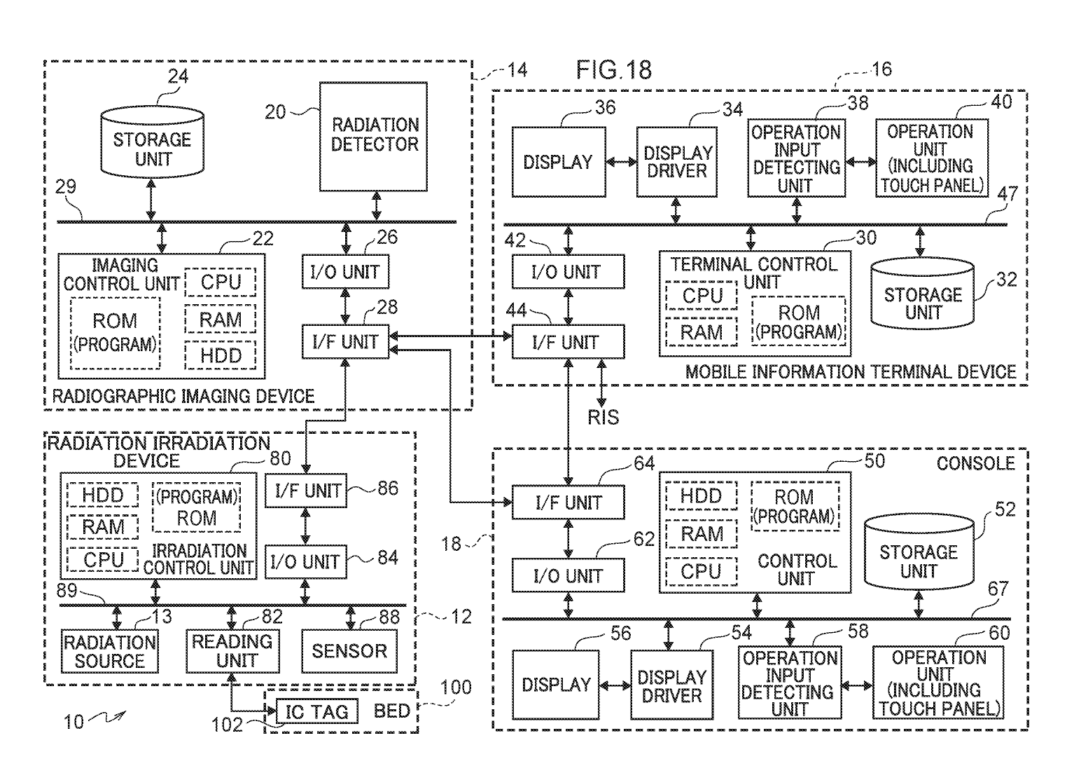

FIG. 18 is a block diagram that illustrates an example of the schematic configuration of the radiation irradiation device 12, the radiographic imaging device 14, the mobile information terminal device 16, and the console 18 of the present embodiment.

As illustrated in FIG. 18, the radiographic imaging device 14 of the present embodiment differs from the radiographic imaging device 14 of the first embodiment only in that the operation detecting unit 23 and the operation button 25 provided in the radiographic imaging device 14 of the first embodiment (see FIG. 2) are omitted.

As illustrated in FIG. 18, the radiation irradiation device 12 of the present embodiment includes the radiation source 13, an irradiation control unit 80, a reading unit 82, an I/O unit 84, an I/F unit 86 and a sensor 88. The radiation source 13, the irradiation control unit 80, the reading unit 82, the I/O unit 84 and the sensor 88 are mutually interconnected via a bus 89 such a as system bus or a control bus, so that various information can be transmitted and received therebetween.

The irradiation control unit 80 includes a CPU, a ROM and a RAM. The CPU controls overall operation of the radiation irradiation device 12. The ROM stores in advance various processing program executed by the CPU. The RAM temporarily stores various data.

The sensor 88 is a sensor that detects a movement of the radiation irradiation device 12. Specific examples of the sensor 88 include velocity sensors and acceleration sensors, but are not limited thereto. Further, in the present embodiment, as the radiation irradiation device 12 is implemented as a visiting car, the radiation irradiation device 12 includes wheels (wheels 122 in FIG. 25) as a movement means. Therefore, the sensor 88 may be a sensor detecting rotation of the wheels, which are the movement means.

Plural beds 100 are equipped in a hospital room or an examination room of a hospital. Therefore, an ID, which is identification information to identify each of the beds 100 is stored in an Integrated Circuits (IC) tag 102. In the present embodiment, the ID of the bed 100 and the subject W (patient) using the bed 100 is associated in advance.

In a case in which the sensor 88 detects that the radiation irradiation device 12 has moved, the reading unit 82 reads the ID stored in the IC tag 102 that is provided in the bed 100. In the present embodiment, each of the beds 100 equipped in a hospital room or an examination room is provided with the IC tag 102 storing an ID for identifying the respective bed 100. As such IC tags 102, IC tags that are attached to the equipments and supplies in the hospital for management thereof may be used. Alternatively, the IC tags 102 used for the present embodiment may be used for management of the equipments.

The ID read by the reading unit 82 is transmitted to the radiographic imaging device 14 through the I/F unit 86. Specifically, a signal indicating the ID is transmitted from the radiation irradiation device 12 to the radiographic imaging device 14; however, the signal indicating the ID is simply referred to as “ID” in the present embodiment for the sake of simplicity of the description. The I/F unit 82 of the radiographic imaging device 14 serves as an example of a receiving unit, and receives the ID transmitted from the irradiation control unit 80 (the radiation irradiation device 12).

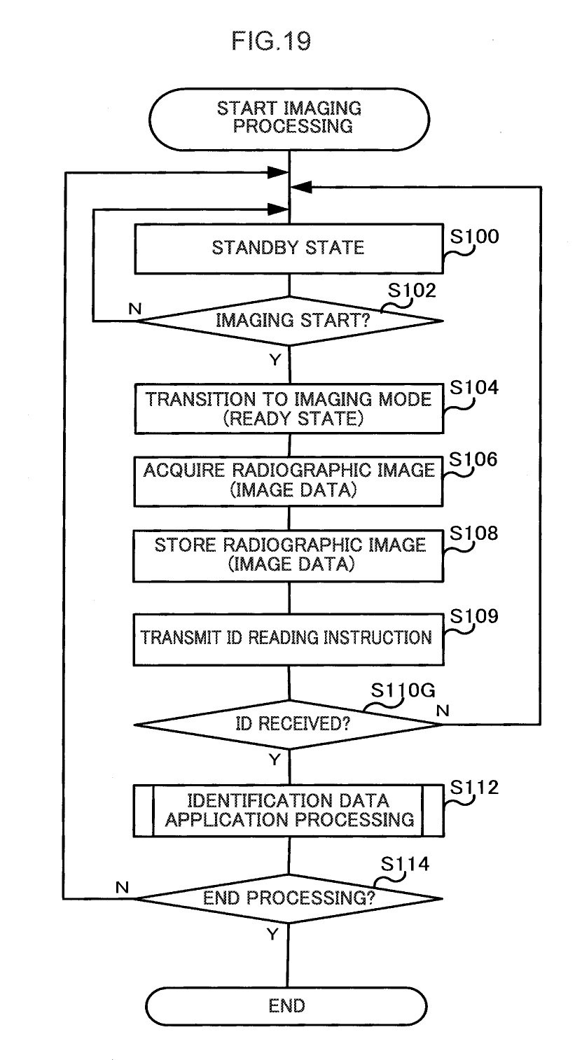

FIG. 19 is a flowchart that illustrates an example of the flow of imaging processing executed by the imaging control unit 22 of the radiographic imaging device 14 according to the present embodiment. The same step numbers are assigned to the steps in FIG. 19 that perform the same processes as in the imaging processing illustrated in FIG. 3, and explanations thereof are omitted.

As illustrated in FIG. 19, the imaging processing of the present embodiment is the same as that of the first embodiment except that Step S110G is performed in place of Step S110A of the imaging processing of the first embodiment.

In Step S110G the imaging control unit 22 determines whether or not an ID is received from the radiation irradiation device 12. In a case in which the determination is negative in Step S110G the imaging control unit 22 returns the process to Step S100. In a case in which the determination is affirmative in Step 110G the process proceeds to Step S112.

In the identification data application processing (see FIG. 4) of the present embodiment, it is preferable to incorporate the received ID in the dummy data in Step S150, in which the dummy data is generated. Due to the dummy data including the ID for identifying the bed 100, the console 18 may associate the subject information with image data of a radiographic image based on the ID, thereby enables easy association.

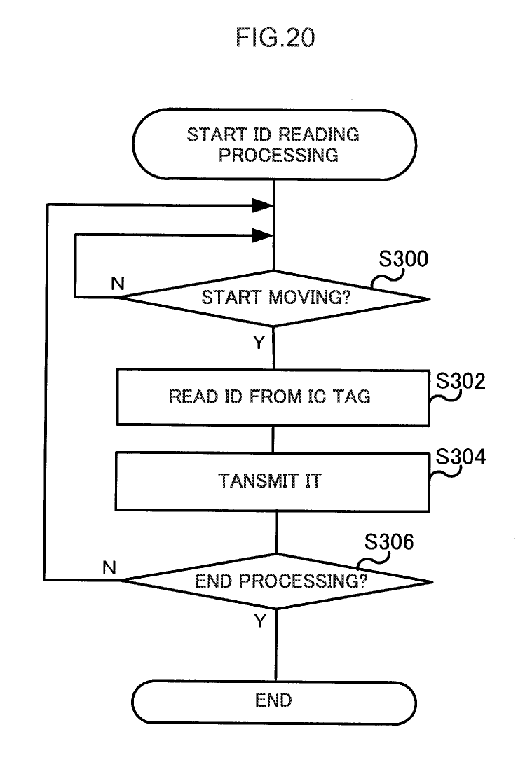

The radiation irradiation device 12 executes ID reading processing as illustrated in FIG. 20.

In Step S300, the irradiation control unit 80 determines whether or not the radiation irradiation device 12 has started to move. In a case in which it is determined that the there is no movement based on the detection result of the sensor 88, the determination is negative.

However, in a case in which it is determined that the radiation irradiation device 12 has started to move, the determination is affirmative and the process proceeds to Step S302. In Step S302, the irradiation control unit 80 reads the ID from the IC tag 102 using the reading unit 82.

In next Step S304, the irradiation control unit 80 transmits the read ID to the radiographic imaging device 14 through the I/F unit 86.

In next Step S306, determination is made as to whether or not to terminate the ID reading processing. For example, in a case in which the radiation irradiation device 12 is still turned on, the determination is negative, the process returns to Step S300 and the ID reading processing is repeated. However, in a case in which the radiation irradiation device 12 is turned off, the ID reading processing ends.

In this way, in the present embodiment, in a case in which the radiation irradiation device 12 is moved from the bed after imaging of the subject W on the bed 100, the radiation irradiation device 12 reads the ID from the IC tag 201 of the bed and transmits the ID to the radiographic imaging device 14. In a case in which the imaging control unit 22 of the radiographic imaging device 14 determines that an ID is received, the identification data is applied to the image data of the radiographic image and the image data is stored in the storage unit 24.

The association processing executed by the console 18 according to the present embodiment is similar to the association processing (see FIG. 6) of the embodiments described above and, thus, description thereof will be omitted.

In this way, similarly to the console 18 of the embodiments described above, the console 18 may easily associate subject information with image data of a radiographic image. Accordingly, the subject W and the image date of the corresponding radiographic image can be easily associated.

In the present embodiment, the radiation irradiation device 12 reads an ID from the IC tag 102 in a case in which the radiation irradiation device 12 moves away from the bed 100. However, the device for reading an ID is not limited thereto. For example, the radiographic imaging device 14 may include the reading unit 82 or the sensor 27 described above, and may read an ID from the IC tag 102 in a case in which the sensor 27 detects the movement.

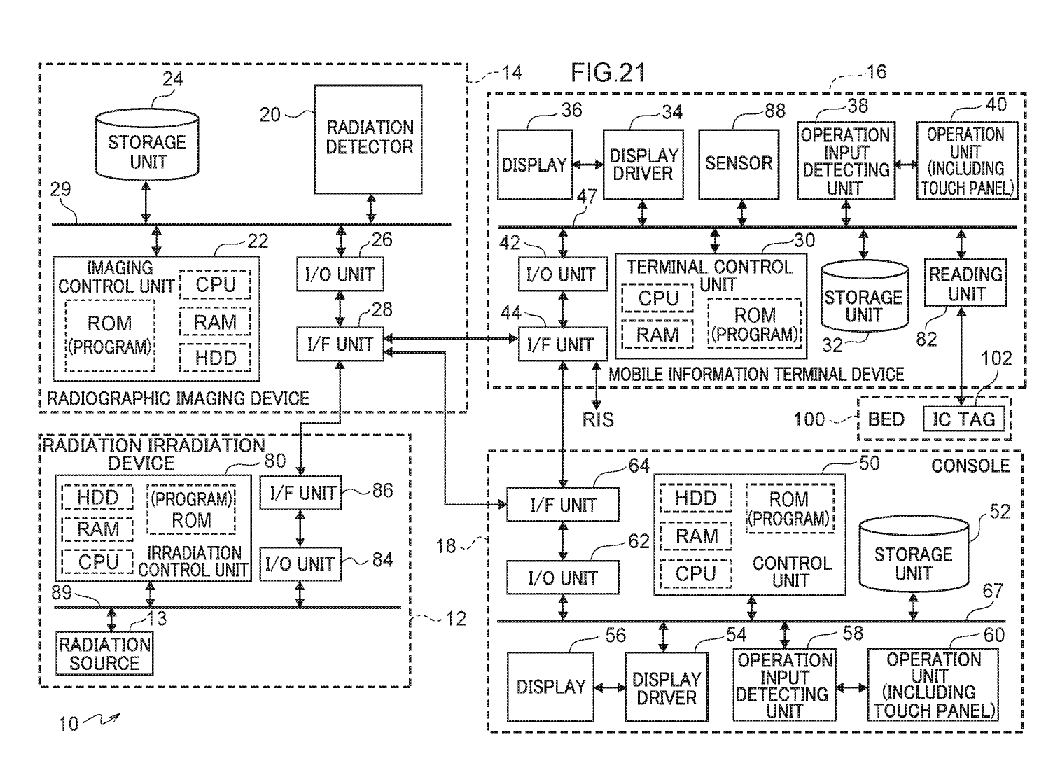

Alternatively, for example, the mobile information terminal device 16 may include the reading unit 82 and the sensor 88, as illustrated in FIG. 21, and may read an ID from the IC tag 102 in a case in which the sensor 88 detects the movement.

In the present embodiment, an ID is read from the IC tag 102 when the device separates away from the bed 100. However, the timing to read an ID is not limited thereto, and am ID may be read from the IC tag 102 in a case in which the device approaches the bed 100. In this case, for example, the reading unit 82 may read the ID from the IC tag 102 at a predetermined time interval. Since the read ID changes when the device moves and approaches another bed 100, the ID may be acquired from the IC tag 102 in a case in which the read ID changes. Alternatively, the radiation irradiation device 12 may read an ID from the IC tag 102 while the radiographic imaging device 14 is performing imaging of a radiographic image of the subject W, and then, in a case in which the sensor 88 detects a start of movement, the radiation irradiation device 12 may transmit the read ID to the radiographic imaging device 14.

### Seventh Embodiment

In the present embodiment, a case will be described in which the radiographic imaging device 14 gives a notification of application of the identification data to image data.

In the present embodiment, a specific example will be described in which a notification of application of the identification data to image data is provided in the radiographic imaging system 10 of the sixth embodiment.

In the present embodiment, similarly to the sixth embodiment, a specific example will be described in which the radiation irradiation device 12 is a visiting car, and the radiation irradiation device 12 reads information identifying a bed of the subject W and transmits the information to the radiographic imaging device 14.

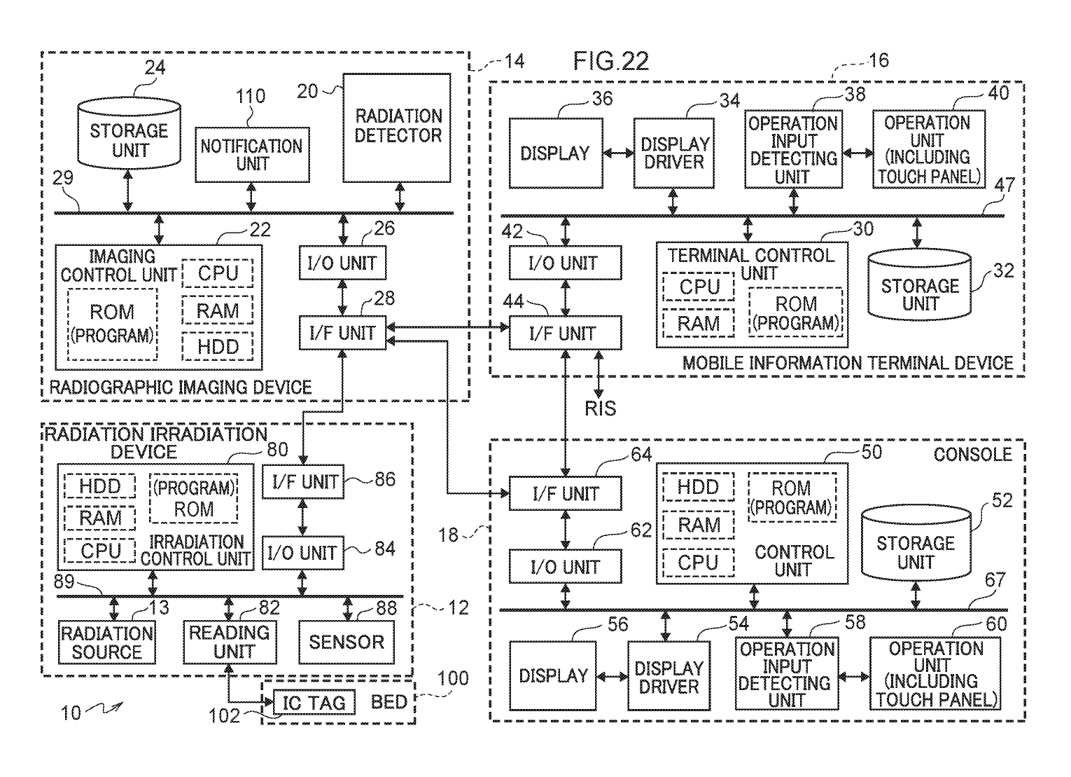

FIG. 22 is a block diagram that illustrates an example of the schematic configuration of the radiation irradiation device 12, the radiographic imaging device 14, the mobile information terminal device 16, and the console 18 according to the present embodiment.

As illustrated in FIG. 22, the radiographic imaging device 14 of the present embodiment differs from the radiographic imaging device 14 of the sixth embodiment (see FIG. 18) only in that it includes a notification unit 110.

The radiographic imaging device 14 of the present embodiment includes, inside the casing 15 illustrated in FIG. 23, the radiation detector 20, the imaging control unit 22, the storage unit 24, the I/O unit 26, and the I/F unit 28. As illustrated in FIG. 23, the casing 15 of the present embodiment is a rectangular casing having a front face as a surface which is irradiated with radiation R, and a back face that is an opposite face to the front face. A side face of the casing 15 includes a slope portion 111 that slopes from the back face toward the front face. In the example of FIG. 23, the notification unit 110 is configured by Light Emitting Diode (LED) lights, which are disposed at respective slope portions at each side, or in the vicinity of respective slope portions.

The notification unit 110 gives notification such that the imaging control unit 22 has applied the identification data. Specifically, the notification unit 110 of the present embodiment gives notification such that the identification data application processing (see FIG. 4) has been executed.

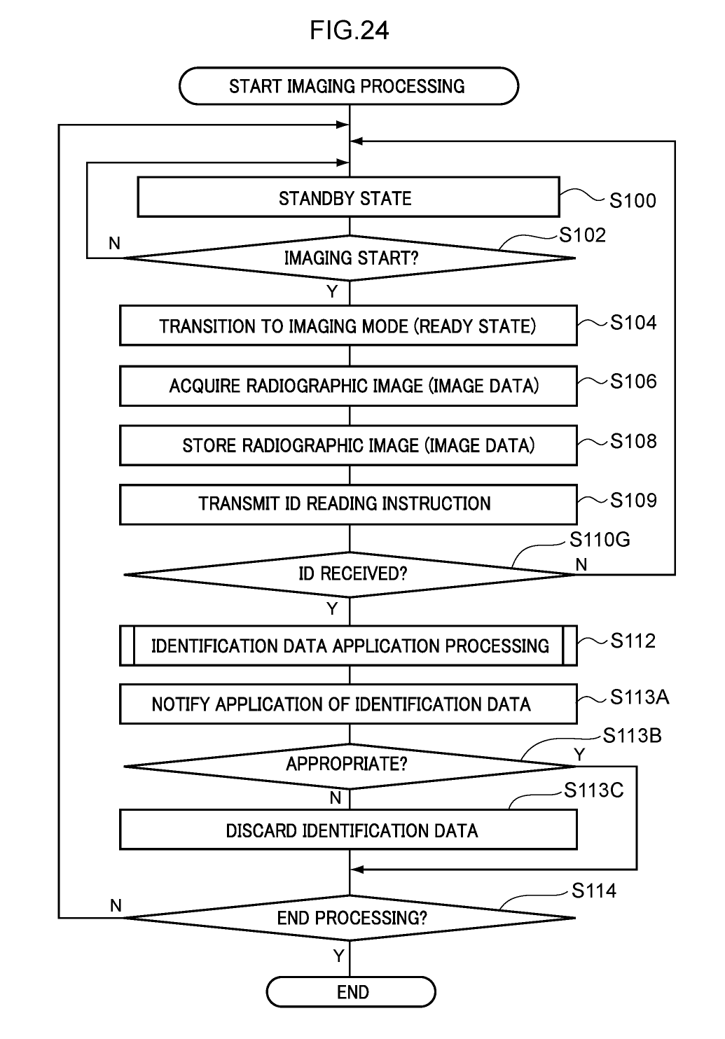

FIG. 24 is a flowchart that illustrates an example of the flow of imaging processing executed by the imaging control unit of the radiographic imaging device 14 according to the present embodiment. The same step numbers are assigned to the steps in FIG. 24 that perform the same processes as in the imaging processing illustrated in FIG. 19, and explanations thereof are omitted.

As illustrated in FIG. 24, the imaging processing is the same as that in the sixth embodiment except that processes of Steps S113A, S113B, S113C are performed after Step S112 of the imaging processing of the sixth embodiment.

In Step S113A, the imaging control unit 22 gives notification of application of the identification data using the notification unit 110. As described above, since the notification unit 110 is configured by the LED lights in the present embodiment, the notification is given by controlling the lightening state of the LED lights according to a predetermined method such as turning on the LED light that has been turned off, lighting the LED light with a predetermined color, blinking the LED light or the like.

In the present embodiment, since a case in which the imaging control unit 22 performs application of the identification data is a case in which the radiation irradiation device 12 moves away from or approaches the bed 100, the radiographic imaging device 14 may often be stored in a storage case of the radiation irradiation device 12. For example, as in the example illustrated in FIG. 25, the radiographic imaging device 14 may be stored in the storage case 120 provided at a side face of a visiting car main body 121. In this case, the radiographic imaging device 14 is generally stored in the storage case 120 so that the front side that is irradiated with the radiation R is facing toward the visiting car main body 121 side. However, in the example illustrated in FIG. 25, since a portion of the radiographic imaging device 14 is exposed from the upper portion of the storage case 120, the notification by the notification unit 110 is visible to a user even in a state in which the radiographic imaging device 14 is stored in the storage case 120.

The configuration is not limited to the example of FIG. 25, and the notification by the notification unit 110 of the stored radiographic imaging device 14 may be made visible to a user by configuring a face of the storage case 120 that is opposite to a face at the visiting car main body 121 side so as to be transparent or suchlike.

The example of FIG. 25 illustrates a state in which one radiographic imaging device 14 is stored in the storage case 120. However, embodiments are not limited thereto and the storage case 120 may store plural radiographic imaging devices 14. Even in a case in which plural radiographic imaging devices 14 are stored in the storage case 120 in a state in which a back face and a front face of the respective radiographic imaging devices 14 are overlaid, as illustrated in FIG. 23, since the notification unit 110 of the present embodiment is disposed at the slope portion 111 at the side face or in the vicinity thereof, the notification is visible to a user. In a case in which plural radiographic imaging devices 14 are stored in the storage case 120, the visibility of the notification by the notification unit 110 can be improved by storing a larger radiographic imaging device 14 (having a large area to be irradiated with the radiation R) at the visiting car main body 121 side.

In the radiographic imaging system 10 of the present embodiment, the user that has viewed the notification by the notification unit 110 may determine whether or not the timing of application of the identification data is appropriate. For example, the imaging control unit 22 may apply the identification data even in cases in which the radiation irradiation device 12 has approached the bed 100 with a mistaken subject W, the radiation irradiation device 12 is moved in order to change the position with respect to the bed 100, or the like. Since these are not proper timing to apply the identification data, the user may determine that the timing of application of the identification data is inappropriate.

In a case in which it is determined that the timing of application of the identification data is inappropriate, the user may perform a predetermined operation through the operation unit 40 of the mobile information terminal device 16. In a case in which the predetermined operation is performed, the terminal control unit 30 transmits to the radiographic imaging device 14 information indicating that the timing of application of the identification data is inappropriate.

At next Step S113B, the imaging control unit 22 determines whether or not the timing of application of the identification data is appropriate. In a case in which the radiographic imaging device 14 has received the information indicating that the timing of application of the identification data is inappropriate, the determination is negative and the process proceeds to Step S113C.

In Step S113C, the imaging control unit 22 discards the identification data that has applied by the identification data application processing at Step S112, and the process proceeds to Step S114.

However, in a case in which the radiographic imaging device 14 does not receive the information indicating that the timing of application of the identification data is inappropriate even after a predetermined time period lapsed, the determination in Step S113B is affirmative and the process proceeds to Step S114.

The association processing executed by the console 18 according to the present embodiment is similar to the association processing (see FIG. 6) of the embodiments described above and, thus, description thereof will be omitted.

In this way, the radiographic imaging device 14 of the present embodiment gives notification of application of the identification data using the notification unit 110, thereby enables the user to be aware of the time when the identification data is applied.

Further, in a case in which the time when the identification data has been applied is inappropriate, the radiographic imaging device 14 discards the identification data that has been applied at the inappropriate time based on the information indicating as such, thereby enables appropriate application of the identification data.

In the present embodiment, explanation has been given in a case in which the notification unit 110 is configured by LED lights; however, embodiments are not limited thereto. For example, the notification unit 110 may be configured by any other unit for visible indication, a unit for audible indication using audio player devices such as a loudspeaker, or a combination of visible indication and audible indication.

In the present embodiment, in a case in which the application time of the identification data is inappropriate, the applied identification data is discarded; however, embodiments are not limited thereto. The identification data may not be discarded, and may be stored in the storage unit 24 or the like with association with information indicating that the application time of the identification data is inappropriate.

In the present embodiment, a case has been described in which the user performs an operation to the operation unit 40 of the mobile information terminal device 16 in a case in which the application time of the identification data is inappropriate. However, the device to which the user's operation is performed is not limited to the information terminal device 16. For example, as in the radiographic imaging device 14 of the first embodiment, the radiographic imaging device 14 may include the operation detecting unit 23 and the operation button 25, and the user may perform operation on the operation button 25.

As described above, the radiographic imaging device 14 according to each of the embodiments described above includes: the radiation detector 20 serving as an imaging unit that images a radiographic image according to irradiated radiation R; the storage unit 24 serving as a storage unit storing image data of plural radiographic images acquired by the radiation detector 20; and the imaging control unit 22 serving as an identification data application unit that stores, in the storage unit 24, the identification data used for partitioning the image data stored in the storage unit 24 between before and after detection of a predetermined condition in a case in which the predetermined condition is detected.

In addition, the console 18 includes: a reading unit that reads the image data and the identification data stored in the storage unit 24 of the radiographic imaging device 14; and the control unit 50 that serves as a controller executing a control for storing the image data and subject information relating to a subject W corresponding to the image data in the storage unit 52 serving as an image storing unit in association with each other based on the identification data.

Thus, according to the radiographic imaging device 14 of each embodiment described above, plural sets of image data acquired by continuous imaging are partitioned by the identification data when the predetermined condition is detected. After the image data is partitioned by the identification data, there are a case in which the imaging of a radiographic image is repeated again and a case in which the imaging process ends. Therefore, by setting the predetermined condition as a condition indicating a change of the subject W, the console 18 can easily associate the subject information and the image data with each other by specifying and using the identification data.

Thus, according to each radiographic imaging device 14 described above, subject information, in other words, a subject W and image data of a radiographic image acquired by imaging the subject W can be easily associated with each other.

Generally, while the number of radiographic images to be imaged of a subject W using order information is determined in advance, there are cases in which the number of images to be imaged set in advance and the number of actually acquired images are different from each other when a re-imaging is performed due to an imaging failure or the like. In addition, there are cases in which additional imaging request (additional order information or the like) is generated separately from the order information acquired in advance by the mobile information terminal device 16 from the console 18 or the like. Further, there are also cases in which the number of images to be imaged set in advance and the number of actually acquired images are different from each other due to a malfunction of the radiation detector 20, a user's erroneous operation, and the like. In such a case, it is not always easy to associate the image data of an acquired radiographic image and the subject information, and such association may not be successfully made depending on the situations.

In contrast to this, in the radiographic imaging device 14 according to each embodiment described above, in a case in which the predetermined condition set is detected by the imaging control unit 22, since the identification data used for partitioning the image data stored in the storage unit 24 between before and after the detection of the predetermined condition is stored in the storage unit 24, the image data and the subject information can be associated with each other by the console 18 by using the identification data. Therefore, also in a case in which the number of images to be imaged set in advance and the number of actually acquired images are different from each other, the subject information and the image data of the corresponding radiographic image can be easily associated by the console 18. In addition, regardless of the presence or absence of the order information, the subject information and the image data of the corresponding radiographic image can be easily associated by the console 18.

In addition, according to each radiographic imaging device 14 described above, even if the subject information is not acquired by the radiographic imaging device 14, the subject information and the image data of the corresponding radiographic image can be easily associated with each other by the console 18.

Furthermore, according to each radiographic imaging device 14 described above, the image data and the identification data are associated inside the radiographic imaging device 14. Thus, the image data and the identification data can be associated with each other without requiring an external device such as the mobile information terminal device 16 or the console 18. In addition, since the association is performed under the control of the imaging control unit 22, the association process can be realized without changing a conventional radiographic imaging device.

In addition, the method for applying the identification data to the image data of a radiographic image is not limited to that of each embodiment described above. For example, a method may be employed in which the identification data is applied to the image data stored in the storage unit 24 in a case in which the radiographic imaging device 14 detects that the device is separate from an imaging stand by a predetermined distance or more by the sensor 27 using a radio frequency identification (RFID). Furthermore, for example, a method may be employed in which an opening/closing sensor detecting the opening/closing of a door is arranged at the door of an imaging room, and the identification data is applied to the image data stored in the storage unit 24 in a case in which the radiographic imaging device 14 detects that the opening/closing sensor detects the opening/closing of the door.

Furthermore, a method may be employed in which a signal is transmitted to the radiographic imaging device 14 in a case in which the radiation irradiation device 12 that is a visiting car, as in the sixth embodiment, is separated from the bed 100 by a predetermined distance or more, and the identification data is applied to the image data stored in the storage unit 24 in response to the reception of the signal at the radiographic imaging device 14.

In addition, for example, a method may be employed in which a weight measuring device is arranged on the floor under an imaging stand, and, in a case in which a decrease in the measured value acquired by the weight measuring device is detected, a determination is made by the radiographic imaging device 14 as to the subject W have been moved from the imaging stand, and the identification data is applied to the image data stored in the storage unit 24.

In the embodiments described above, cases have been described in which the image data and the subject information are associated with each other by a user using the console 18. However, the association method is not limited thereto, and the console 18 may automatically associate the image data and the subject information with each other. As an example of this method, a case will be described hereinafter in which, in a case in which order information including subject information is stored in advance in the storage unit 52 of the console 18, the image data and the subject information are automatically associated with each other by the console 18 based on the stored order information.

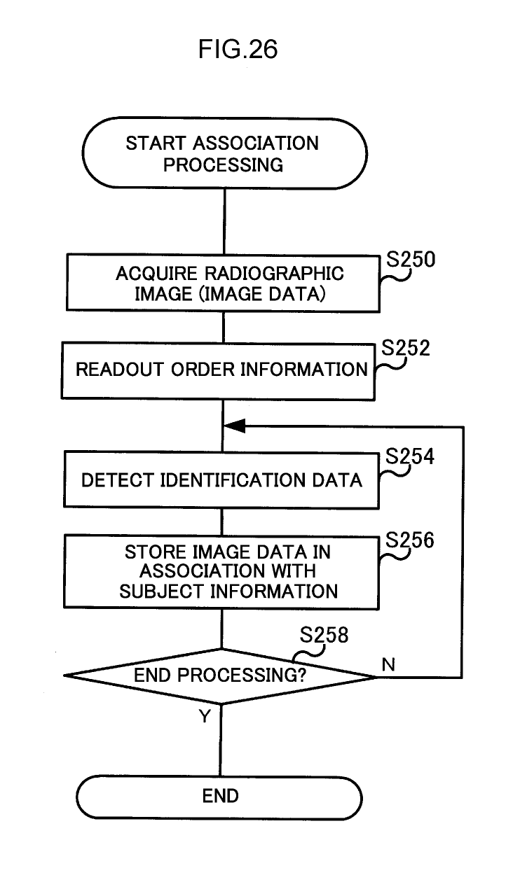

FIG. 26 is a flowchart that illustrates an example of the association processing executed by the console 18 in such a case.

In Step S250 of FIG. 26, similar to Step S200 (see FIG. 6) of the association processing described above, the control unit 50 acquires the image data of a radiographic image from the radiographic imaging device 14 through the I/F unit 64.

In the next Step S252, the control unit 50 reads order information corresponding to the acquired image data from the storage unit 52. A method for identifying order information corresponding to the acquired image data from the order information stored in the storage unit 52 is not particularly limited. For example, in a case in which information (for example, an ID) identifying the radiographic imaging device 14 that has acquired the radiographic image and the sequence of imaging are included in the order information, the ID of the radiographic imaging device 14 may be acquired together with the image data in the acquisition of the image data in Step S250, and the order information may be identified based on the acquired ID and the sequence of imaging.

In the next Step S254, the control unit 50 detects identification data from the acquired image data of the radiographic image. For example, in the case of the first embodiment, the control unit 50 detects dummy data one by one in the order of storage in the storage unit 52.

In the next Step S256, the control unit 50 stores the subject information included in the corresponding order information in the storage unit 52 in association with the acquired image data of the radiographic image.

In the next Step S258, the control unit 50 determines whether or not to end the association processing. In this Step S258, in a case in which there is image data with which subject information is not associated among the sets of image data of the radiographic images acquired in Step S250, the determination is negative and the process returns to Step S254, and the association processing is repeated. In a case in which the acquired sets of image data of all of the radiographic images are associated with the subject information, the determination is affirmative and the association processing ends.

In this way, by executing the association processing by the console 18, the subject information and the corresponding image data can be automatically associated with each other without bothering the user's hand.

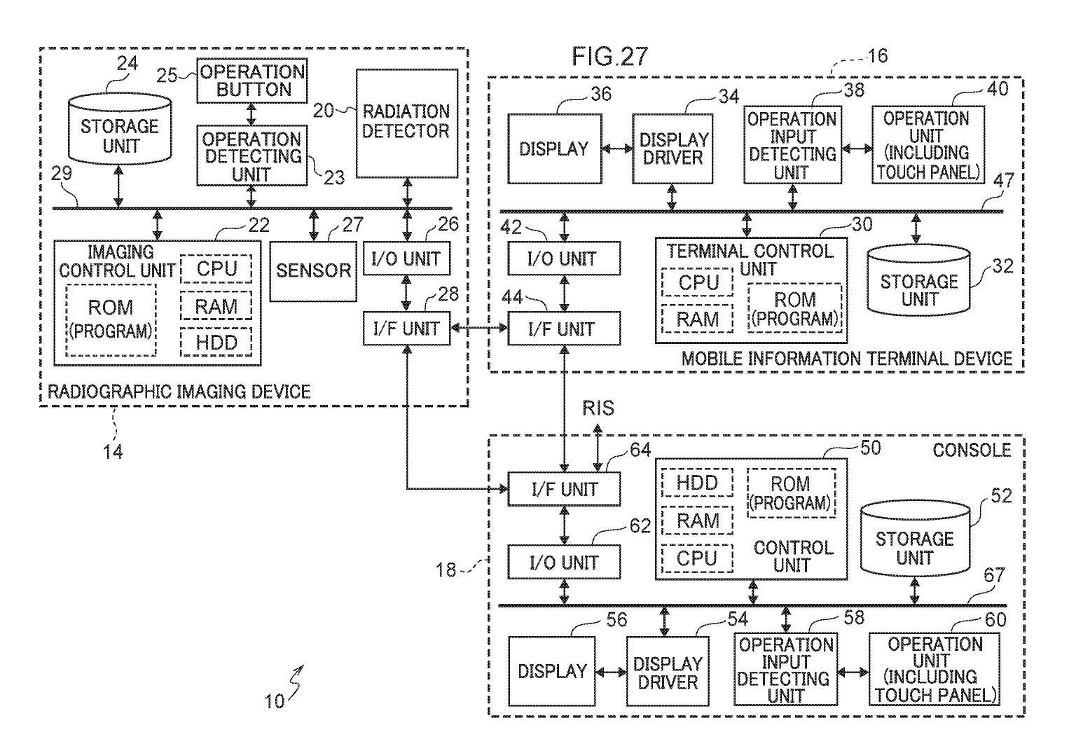

Obviously, the embodiments described above may be appropriately combined. As an example of the combination, the first embodiment and the third embodiment may be combined. FIG. 27 is a block diagram that illustrates an example of the schematic configuration of a radiographic imaging device 14, a mobile information terminal device 16, and a console 18 of such a case. As illustrated in FIG. 27, the radiographic imaging device 14 includes an operation detecting unit 23, an operation button 25, and a sensor 27. In a case in which an operation of the operation button 25 is detected by the imaging control unit 22 or in a case in which the state of the own device is detected to be a predetermined state by the sensor 27, the identification data may be applied.

In the second to seventh embodiments described above, the identification data is applied to the image data after the imaging of the radiographic image. However, the time at which the identification data is applied need not be a time after the imaging. For example, the identification data may be applied to image data stored in the storage unit 24, to which identification data is not yet applied, before imaging of new images.

Furthermore, the radiographic imaging device 14 may detect a time that can be regarded as a time before and after imaging of a radiographic image and apply identification data to the image data acquired (stored in the storage unit 24) during the time period therebetween. For example, in a case of the radiographic imaging device 14 according to the first embodiment, operations of the operation button 25 before and after the imaging processing may be detected, and the identification data may be applied to the image data stored in the storage unit 24 during that time period.

In addition, the radiographic imaging device 14 may store other information in the storage unit 24 in association with the image data together with the identification data. Such information may include, for example, information representing at least one of imaging date or time, time at which the identification data is applied, an elapsed time, and the like. In addition, in the radiographic imaging device 14 of the third embodiment, information based on a detection result detected by the sensor 27 (for example, in Example 3-2, the posture of the radiographic imaging device 14) may be included as the other information.

In the embodiments described above, cases in which the console 18 perform association of the image data and the subject information; however, the association may be performed by a device other than the console 18. For example, the mobile information terminal device 16 may serve as a control device and may perform association of the image data and the subject information.

In the embodiments described above, cases have been described in which the console 18 stores the image data and the subject information in the storage unit 52 in association with each other. However, the location at which such information is stored is not limited to the storage unit 52. For example, such information may be stored in an external storage device (picture archiving and communication system: PACS) or the like.

The radiographic imaging device 14 may include a display unit such as a liquid crystal display. In such a case, the imaging of radiographic images is completed, information representing the completion of the imaging may be displayed on the display unit.

The subject W may not be a human but may be a living thing such as an animal other than a human or a plant or any other subject.

The radiation R used for imaging a radiographic image are not particularly limited and, X rays, γ rays, or the like may be applied.

The configurations and the operations of the radiographic imaging device 14, the console 18, and the like described in the embodiments are merely examples, and obviously the configurations and the operations may be changed within a range not departing from the concept of the present disclosure depending on the situations.

What is claimed is:

1. A radiographic imaging device, comprising:
an imaging unit that is configured to acquire a radiographic image in accordance with irradiated radiation;
a storage unit that is configured to store sets of image data for a plurality of radiographic images acquired by the imaging unit; and
an identification data application unit that is configured to store identification data in the storage unit in a case in which a predetermined condition is detected, the identification data being used for partitioning, between before and after the detection of the predetermined condition, the sets of image data stored in the storage unit.

2. The radiographic imaging device according to claim 1, wherein, in a case in which the predetermined condition is detected, the identification data application unit is configured to group, as the same group, one or more sets of image data that have been stored in the storage unit until the predetermined condition is detected and that have not been previously grouped, and to store, in the storage unit, a different item of the identification data for each group in association with the sets of image data.

3. The radiographic imaging device according to claim 1, wherein the identification data is dummy data of the image data.

4. The radiographic imaging device according to claim 3, wherein the identification data application unit is configured to store the dummy data of the image data at a position partitioning the sets of image data in the same group in a storage area of the storage unit.

5. The radiographic imaging device according to claim 1, wherein the identification data is information that is different from subject information relating to a subject of which a radiographic image is acquired by the imaging unit.

6. The radiographic imaging device according to claim 1, further comprising an operation unit that is configured to be operated by a user, wherein the predetermined condition is a condition indicating that the operation unit has been operated.

7. The radiographic imaging device according to claim 1, further comprising a detection unit that is configured to detect a predetermined state of the imaging device, wherein the predetermined condition is a condition indicating that the predetermined state has been detected by the detection unit.

8. The radiographic imaging device according to claim 7, wherein:
the detection unit is a sensor detecting a movement state of the imaging device; and
the predetermined state is a state acquired by the sensor, indicating that the imaging device is moved by a predetermined distance or more.

9. The radiographic imaging device according to claim 7, wherein:
the detection unit is a sensor that detects a change in a posture of the imaging device; and
the predetermined state is a state acquired by the sensor, in which the posture of the imaging device has changed from a first state to a second state.

10. The radiographic imaging device according to claim 7, wherein:
the detection unit is a sensor that detects pressure applied to the imaging device; and
the predetermined state is a state acquired by the sensor, in which the pressure applied to the imaging device has changed by a predetermined amount or more.

11. The radiographic imaging device according to claim 7, wherein:
the detection unit is a reception unit that receives a signal indicating an approach to at least one selected from the group consisting of a predetermined device and predetermined equipment; and
the predetermined state is a state in which the reception unit has received the signal.

12. The radiographic imaging device according to claim 7, wherein:
the detection unit is a sensor that detects an approach to at least one selected from the group consisting of a predetermined device and predetermined equipment; and
the predetermined state is a state indicating detection of an approach to the at least one selected from the group consisting of the predetermined device and the predetermined equipment.

13. The radiographic imaging device according to claim 7, wherein:
the detection unit is a reception unit that receives a signal indicating a separation from at least one selected from the group consisting of a predetermined device and predetermined equipment; and
the predetermined state is a state in which the reception unit has received the signal.

14. The radiographic imaging device according to claim 7, wherein:
the detection unit is a sensor that detects a separation from at least one selected from the group consisting of a predetermined device and predetermined equipment; and
the predetermined state is a state indicating detection of a separation from the at least one selected from the group consisting of the predetermined device and the predetermined equipment.

15. The radiographic imaging device according to claim 1, further comprising a power source unit to which source power is externally supplied, wherein the predetermined condition is a condition corresponding to source power externally supplied to the power source unit.

16. The radiographic imaging device according to claim 1, wherein the predetermined condition is a condition corresponding to a position between a radiation irradiation device that irradiates the subject with radiation and at least one selected from the group consisting of a predetermined device and predetermined equipment.

17. A radiographic imaging system, comprising:
the radiographic imaging device according to claim 1; and
a control device that includes:
a reading unit that is configured to read the image data and the identification data stored in the storage unit of the radiographic imaging device; and
a control unit that is configured to effect control of storing the image data and subject information relating to a subject corresponding to the image data in an image storing unit in association with each other based on the identification data.

18. The radiographic imaging system according to claim 17, wherein the control device further includes:
a display unit that displays one or more sets of image data of a group partitioned by the identification data so as to be grouped for each group; and
a reception unit that receives the subject information relating to the subject corresponding to the image data displayed on the display unit, and
wherein the control unit is configured to effect control of storing the subject information received by the reception unit in the image storing unit in association with the corresponding image data.

19. The radiographic imaging system according to claim 17, wherein the control unit is configured to acquire the subject information from order information of the radiographic image corresponding to the image data partitioned by the identification data, and to effect control of storing the acquired subject information in the image storing unit in association with the image data.

20. An identification data application method causing a computer to execute a process comprising:
acquiring a radiographic image in accordance with irradiated radiation by using an imaging unit;
storing sets of image data of a plurality of radiographic images acquired by the imaging unit in a storage unit; and
storing identification data in the storage unit in a case in which a predetermined condition is detected, the identification data being used for partitioning, between before and after the detection of the predetermined condition, the sets of image data stored in the storage unit.

21. A non-transitory storage medium storing a program that causes a computer to execute an identification data application process, the identification data application process comprising:
acquiring a radiographic image in accordance with irradiated radiation by using an imaging unit;
storing sets of image data of a plurality of radiographic images acquired by the imaging unit in a storage unit; and
storing identification data in the storage unit in a case in which a predetermined condition is detected, the identification data being used for partitioning, between before and after the detection of the predetermined condition, the sets of image data stored in the storage unit.
